(19) United States
(12) Patent Application Publication
(10) Pub. No.: US 2026/0347829 A1
(43) Pub. Date: Nov. 5, 2026

(54) METHOD TO GENERATE SAFER BIOTECHNOLOGIES FOR CRISPR-BASED GENE EDITING AND THERAPEUTICS

(71) Applicant: THE UNIVERSITY OF NORTH CAROLINA AT GREENSBORO, Greensboro, NC (US)

(72) Inventors: Eric Josephs, Chapel Hill, NC (US); Ashley Herring Nicholas, Greensboro, NC (US); Hillary Dimig, Greensboro, NC (US); Miranda Roesing, Surfside Beach, SC (US)

(21) Appl. No.: 19/117,911

(22) PCT Filed: Oct. 9, 2023
(86) PCT No.: PCT/US2023/076357

§ 371 Date: Apr. 2, 2025

Related U.S. Application Data
(60) Provisional application No. 63/414,112, filed on Oct. 7, 2022.

(51) Int. Cl. C12N 15/10; C12N 1/20; C12N 9/22; C12N 15/113; C12N 15/63; C12N 15/70; C12R 1/19; C40B 40/06

(52) U.S. Cl. CPC C12N 15/102; C12N 1/20; C12N 9/226; C12N 15/113; C12N 15/635; C12N 15/70; C40B 40/06; C12N 2310/20; C12N 2310/531; C12N 2800/101; C12R 2001/19

(57) ABSTRACT

In general, the present disclosure is directed to methods and systems for selecting and/or designing crRNA oligonucleotide sequences which display lower promiscuity for inducing off-target mutations in DNA. The example methods and systems utilize a CRISPR effector system to induce selective damage in oligonucleotide sequences included in a multi-plasmid system.

### RELATED APPLICATION DATA

The present application claims priority pursuant to 35 U.S.C. § 119 (e) to U.S. Provisional Patent Application No. 63/414,112 filed Oct. 7, 2022 which is incorporated herein by reference in its entirety.

### STATEMENT REGARDING FEDERALLY SPONSORED RESEARCH

This invention was made with government support under contract nos. 1 R35G M, 133483-01, and 12718261 awarded by National Institute of Health, National Institute of General Medical Sciences, respectively. The government has certain rights in the invention.

### SEQUENCE LISTING

An electronic sequence listing (060026-00064.xml; size 117 KB; date of creation Oct. 4, 2023) submitted herewith is incorporated by reference in its entirety.

### FIELD

The present disclosure relates to methods for selecting and/or designing crRNAs for use in applications such as gene editing to mitigate off-target mutation.

### BACKGROUND

Class II CRISPR effectors like Cas9, Cas12, and Cas13, are endonucleases that use a modular segment of their RNA cofactors known as CRISPR RNAs (crRNAs) or guide RNAs (gRNAs) to recognize and trigger the degradation of nucleic acids with a sequence complementary to that segment (FIG. 1) These diverse enzymes are derived from a bacterial and archaeal defensive response to invasive plasmids and viruses and, because of their ability to be easily redirected to nucleic acid with different sequences by simply changing the sequence composition of that short portion of their gRNAs called their ‘spacer,’ they have been re-appropriated over the past several years for a number of different biotechnological applications, most notably in precision gene editing. During precision gene editing, a CRISPR effector is transfected into a human cell and directed to introduce a double strand break (DSB) into the genomic DNA at a specific targeted sequence; genomic mutations have been introduced at those sites as a result of mutagenic DSB repair. These technologies have experienced widespread adoption for biomedical research and possess a number of emerging therapeutic applications as well.

### SUMMARY

Generally, the present disclosure is directed to methods and systems for selecting and/or designing crRNA oligonucleotide sequences which display lower promiscuity for inducing off-target mutations in DNA. The example methods and systems utilize a CRISPR effector system to induce selective damage in oligonucleotide sequences included in a multi-plasmid system.

One aspect of example methods and systems includes an organism for hosting the selection. In one example implementation, the organism is a bacterium such as E. coli.

In some example implementations of the disclosure, the crRNA oligonucleotide sequences to be selected for are encoded on separate plasmids which are introduced (e.g., transfected) into the organism. Each separate plasmid also includes an off-target sequence. In this manner, reading of the separate plasmids by the bacterium transcription and/or translation machinery will produce the crRNA oligonucleotide sequences and their affinity for off-target sequences will over time select for crRNA oligonucleotide sequences that display low promiscuity, for example by degrading plasmids having off-target sequences for which the crRNA displays affinity/binds.

Additionally, to select for crRNA nucleotide sequences that display affinity for a target sequence, a target plasmid, including the target oligonucleotide sequence and encoding a toxin is transfected or otherwise present in the bacterium. Based on this design, crRNA sequences which are able to induce breaks in the target plasmid will prevent expression of the toxin, leading to survival of the bacterium.

Thus the combination of a library of plasmids encoding different crRNA sequences and a target plasmid including the target oligonucleotide sequence can be used to select for crRNA which are effective at degrading a target oligonucleotide sequence and therefore preventing expression of the toxin. Concurrently, the combination of the crRNA with an off-target sequence can be used to select for crRNA sequences that display low promiscuity for the off target sequence.

Another aspect of example implementations includes a CRISPR effector. Many endonucleases and variants have been developed to effect gene editing. For implementations of the disclosure, the CRISPR effector is used to induce damage in a double stranded DNA plasmid. Any CRISPR effector which can induce such DNA damage can be used. To include the CRISPR effector in example methods and systems, the CRISPR effector may be encoded in an effector plasmid, which is transduced or otherwise present in the bacterium.

In one example selection assay, the separate plasmids encoding the crRNA oligonucleotide sequences are transfected into a bacterium, where the bacterium includes a first plasmid having a target oligonucleotide sequence and the first plasmid also encodes a toxin.

As used herein, oligonucleotides sequences that “encode” a product have sequences whereupon transcription/translation by the bacterium machinery, produce the product (e.g., the toxin and/or an RNA sequence).

The present disclosure is directed to methods of crRNA design and nucleic acid sequences derived therefrom. In particular, the present disclosure provides methods for designing and/or selecting oligonucleotide sequences displaying reduced promiscuity for off-target mutation in gene editing applications using a CRISPR effector system.

An example aspect of the present disclosure can include a selection assay for plasmids encoding crRNA sequences. In an example implementation, the plasmids include an oligonucleotide sequence which upon transcription produces a crRNA having the general sequence x-gRNA, where x is a sequence of n-m nucleotides. In some example implementations, the sequence x is capable of forming a secondary structure (e.g., hairpin) with the gRNA portion of the x-gRNA. In other implementations, the sequence x can be determined at random (e.g., using a recombinant library). The gRNA portion can include a sequence of nucleotides complementary to at least a portion of a target sequence (e.g., a mutant or mutated gene).

As used herein, complementary nucleotides are based on Watson-Crick base paring rules. More particularly, Adenine (A) can form a base pair with Thyamine (T), and Guanine (G) forms a base pair with Cytosine (C). For RNA bases, Thyamine is replaced by uracil (U). Using this framework, a complementary sequence to ATGC would be TACG for DNA and UACG for RNA.

In some example implementations, the plasmids encoding crRNA sequences can each have the same gRNA portion while having different x sequences. In this manner, the assay can be used to determine a leading oligonucleotide sequence x that can reduce promiscuity of the gRNA for off-target mutations.

Alternatively, in certain implementations, the plasmids encoding gRNA sequences can each have different gRNA portions while having the same x sequences.

In one example implementation, the plasmid selection may be determined iteratively. For instance, a process according to the present disclosure can include determining an x sequence by conducting a selection assay where the crRNA sequences have the same gRNA. As the plasmids encoding the crRNA are selected for both efficacy and reduced promiscuity, the remaining plasmids (e.g., after a certain number of bacterium growth cycles) can be isolated and sequenced to determine a selected x oligonucleotide sequence. A second selection assay can be performed to determine a gRNA sequence by conducing the assay where the crRNA sequences each include the selected x oligonucleotide sequence and while including different gRNA sequences. As should be understood, this process can be performed in any order (e.g., to determine a selected gRNA sequence then a selected x oligonucleotide sequence or vice versa).

An example embodiment of the present disclosure can also include a process for crRNA selection. One aspect of the process can include identifying a target genetic sequence. The target genetic sequence can be selected from any organism. In a preferred embodiment, the target genetic sequence is selected from a mammal. In a further preferred embodiment, the target genetic sequence is selected from a mutation or variant sequence that results in phenotypic abnormality. For instance, the target genetic sequence can include a disease (e.g., cancer or sickle cell disease) causing and/or promoting sequence.

Another aspect of an example selection process can include obtaining a first plasmid including the target genetic sequence, where the first plasmid further includes an oligonucleotide sequence encoding a toxin.

A still further aspect of the example selection process can also include obtaining a second plasmid, where the second plasmid includes an oligonucleotide sequence encoding a CRISPR effector system (e.g., cas9).

The example selection process can also include identifying one or more off-target sequences (e.g., healthy alleles).

In an example selection process, the process may include obtaining a plurality of third plasmids, each of the third plasmids including a distinct off-target sequence of the one or more off-target sequences, and where the third plasmid includes an oligonucleotide sequence further encoding a crRNA sequence.

In some implementations, the selection assay can also include transfecting the organism with the first plasmid, the second plasmid, and/or the plurality of third plasmids.

According to embodiments of the present disclosure, the crRNA sequence can comprise an x-gRNA structure including an ‘x’ oligonucleotide portion covalently attached to a gRNA oligonucleotide portion, wherein the gRNA oligonucleotide portion is an oligonucleotide sequence substantially complementary to a portion of the target oligonucleotide sequence.

In some implementations identifying the target sequence can include determining a sequence position for each of one or more protospacer motifs based at least in part on the CRISPR effector, were each of the one or more protospacer motifs include an adjacent sequence of nucleotides; and assigning at least one sequence position as a protospacer position; and identifying the two or more target sequences as a sequence of nucleotides immediately downstream (toward the 3′ end) of the protospacer position.

For certain example methods the CRISPR effector can be enAsCas12a. For certain example methods the CRISPR effector can be Cas9.

In some example methods the one or more protospacer motifs are from the group consisting of: NGG, NAG, TTYN, CTTV, RTTC, TATM, CTCC, TCCC, TACA, RTTS, TATA, TGTV, ANCC, CVCC, TGCC, GTCC, TTAC, or combinations thereof.

In some example methods, the one or more protospacer motifs are from the group consisting of: NGG, NAG, TTYN, CTTV, RTTC, TATM, CTCC, TCCC, TACA, or combinations thereof.

In some example methods, the CRISPR effector is Cas9 and the one or more protospacer motifs are from the group consisting of: NGG, NAG, or combinations thereof

### BRIEF DESCRIPTION OF THE FIGURES

FIG. 1 illustrates a diagram depicting how CRISPR effectors recognize DNA complementary to their RNA cofactors to introduce double strand breaks (DSBs) at those sites. Genetic mutations result from non-conservative repair of those sites.

FIG. 2 illustrates a diagram depicting crRNA structures in combination with a CRISPR effector to show extending the crRNA (x-gRNA) outside the DNS-targeting region (gray “spacer”) can introduce internal interactions that inhibit full invasion of off-target sequences.

FIG. 3 illustrates a graph (left) and an image of a predicted secondary structure of the crRNA (right). The x-gRNA with unusual predicted structure performs best.

FIG. 4 illustrates a schematic depicting an example embodiment of the disclosure. X-gRNA libraries are co-transformed with a toxic plasmid and x-gRNA screened for cleavage activity (vs. a toxic plasmid with target site) and specificity (no activity vs. off-target on the guide plasmid). (Right). Only active and specific x-gRNAs will survive for rescreening and analysis.

FIG. 5 illustrates images of using the regular EMX1 sgRNA, Cas9 is active and can cleave toxin plasmid: bacteria survive (activity positive selection, left column images). If there is a known EMX1 of/target introduced on the guide plasmid, self-cleavage will cause the bacteria to die (specificity negative selection, right column images).

FIG. 6 illustrates a gel displaying three randomly picked colonies from the EMX1 x-gRNA library after screening (as in FIG. 5, top right image) were sequenced. The x-gRNA from those colonies were synthesized to test Cas9 cleavage activity vs DNA with EMX1 target (ON) and a known off-target (OFF1) in vitro. All three screened x-gRNAs exhibited strong activity vs. on-target DNA (comparable to sgRNA) but abolished activity at the off-target target that only differs by 2 PAM-distal nucleotides.

FIG. 7 illustrates a graph displaying in vitro cleavage activity of the top five extended gRNAs generated from the invention and their activity at the target site and four sites where the regular gRNA is known to exhibit significant off-target activity. N=3 independent trials.

FIG. 8 illustrates a graph depicting the ratio of Cas9 activity at oncogenic mutations (KRAS G12D) vs healthy gene sequence (KRAS WT) based on data from Table 1.

FIG. 9A is a schematic of the process of the present invention. SECRETS screen is provided identify short 5′-nucleotide extensions to gRNAs that increase Cas9 gene editing specificity for a specific target with known off-targets. Simplified schematics of the three SECRETS plasmids: (i) high-copy number plasmid with inducible toxin and target sequence of interest to select for “on-target” activity, (ii) medium copy plasmid for inducible expression of Cas9, and (iii) low-copy plasmid for gRNA expression and counterselection for “off-target” activity with kanamycin resistance cassette and a known “off-target” sequence for the gRNA.

FIG. 9B is a schematic of the process of the present invention. SECRETS screen is provided identify short 5′-nucleotide extensions to gRNAs that increase Cas9 gene editing specificity for a specific target with known off-targets. (Upper) a standard gRNA has 20 nt “spacer” (DNA-targeting) segment while (lower) an extended gRNA (x-gRNA) has an additional 8-12 nt to the 5′-of the spacer.

FIG. 9C is a schematic of the process of the present invention. SECRETS screen is provided identify short 5′-nucleotide extensions to gRNAs that increase Cas9 gene editing specificity for a specific target with known off-targets. During SECRETS selection, E. coli expressing standard gRNAs that exhibit promiscuous activity are unable to survive.

FIG. 9D is a schematic of the process of the present invention. SECRETS screen is provided identify short 5′-nucleotide extensions to gRNAs that increase Cas9 gene editing specificity for a specific target with known off-targets. Members of large, randomized x-gRNA libraries can be screened simultaneously using SECRETS for 5′-extensions that result in efficient on-target Cas9 activity and minimized off-target activity that can be used for highly-specific gene editing applications at that target.

FIG. 10A are images depicting results of the SECRETS method. SECRETS is highly selective for on-target activity and counter-selective for off-target activity. SECRETS screen with pSECRETS-A, an empty toxin plasmid (pSECRETS-C with no target), and pSECRETS-B with an EMX1 sgRNA after plating on non-selective (left) vs selective (right) conditions.

FIG. 10B are images depicting results of the SECRETS method. SECRETS is highly selective for on-target activity and counter-selective for off-target activity. SECRETS screen with pSECRETS-A, pSECRETS-C with EMX1 ON, and pSECRETS-B with an EMX1 sgRNA on non-selective (left) vs selective (right) conditions.

FIG. 10C are images depicting results of the SECRETS method. SECRETS is highly selective for on-target activity and counter-selective for off-target activity. SECRETS screen with pSECRETS-A, pSECRETS-C with EMX1 ON, and pSECRETS-B with an EMX1 sgRNA and EMX1 OFF1 on non-selective (left) vs selective (right) conditions.

FIG. 10D are images depicting results of the SECRETS method. SECRETS is highly selective for on-target activity and counter-selective for off-target activity. SECRETS screen with pSECRETS-A, pSECRETS-C with EMX1 ON, and pSECRETS-B with an EMX1 x-gRNA library (N8) and EMX1 OFF1 on non-selective (left) vs selective (right) conditions.

FIG. 11A is a graphical representation of the selection and validation of x-gRNAs. The top five x-gRNAs recovered from two SECRETS screen replicates for the EMX1 gRNA were identified via next-generation sequencing (NGS) for in vitro validation.

FIG. 11B is a graphical representation of the selection and validation of x-gRNAs. All five x-gRNAs exhibit remarkable on-target activity (comparable or greater than “enhanced specificity” Cas9 variant eCas9) and greatly reduced off-target activities across four known off-target sites for the standard x-gRNA during in vitro cleavage assays using purified Cas9 and in vitro-transcribed (x-)gRNAs. n=3 replicates each. Error bars=standard error of the mean.

FIG. 11C is a graphical representation of the selection and validation of x-gRNAs. Highly-specific x-gRNAs could also be readily identified from SECRETS screens for gRNAs for FANCF and VEGFA targets. n≥4 each. Error bars=standard error of the mean.

FIG. 11D is a schematic representation of the selection and validation of x-gRNAs. CHANGE-seq5 reveals that x-gRNAs identified through SECRETS exhibit minimal and greatly-reduced genome-wide off-target activity, with no novel off-target activity identified relative to the standard gRNA. n=2 biological replicates each.

FIG. 11E is a schematic and graphical representation of the selection and validation of x-gRNAs. x-gRNAs identified through SECRETS exhibit robust gene editing activity following transfection of purified Cas9 ribonucleoprotein (RNP) complexes. sg=standard gRNA, dCas9=catalytically inactive Cas9, eCas9=enhanced specificity Cas9, NGS=next-generation (illumina) sequencing, T7E1=T7E1 mutation detection assay. n=3 biological replicates, 2 NGS replicates, and 3 T7E1 replicates. Error bars=binomial confidence (NGS) and standard error of the mean (T7E1).

FIG. 12 are representative gel images of Cas9 RNP cleavage activity with EMX1 sgRNA and x-gRNAs.

FIG. 13A is a representation of a gel image showing trials using FANCF.

FIG. 13B is a representation of a gel image showing trials using VEGF.

FIG. 13C is a graphical representation of in vitro cleavage of FANCF target and top off-target sites by Cas9 and eCas9 using WT and hairpin gRNAs.

FIG. 13D is a graphical representation of in vitro cleavage of VEGF target and top off-target sites by Cas9 and eCas9 using WT and hairpin gRNAs.

FIG. 14A is a schematic representation of modifications to the SECRETS plasmids to expand the 5′-extension space and selection strength. Examples of potential modifications to the SECRETS plasmids.

FIG. 14B is a gel image of modifications to the SECRETS plasmids to expand the 5′-extension space and selection strength. EMX1 x-gRNA libraries with 8 random nucleotides and 4 potential (tetraloop) sequences between N8 and the spacer (UUCG, GCAA, CUUG, or no tetraloop sequence) were screened against EMX1 OFF1 using SECRETS assay—4\hat{ }8*4=262, 144 possible 5′-extensions. 3 colonies of the survivors were picked at random and sequenced using colony PCR and Sanger sequencing then characterized using in vitro cleavage assays of purified RNPs, and all 3 exhibited extremely strong on-target activity (comparable to sgRNA) and essentially no off-target activity at EMX1 OFF1. EMX1 sgRNA is SEQ ID NO: 105—GAGUCCGAGCAGAAGAAGAA; EMX1 xt1-gRNA is SEQ ID NO: 106—UUGAGUAUUUCGGAGUCCGAGCAGAAGAAGAA; EMX1 xt2-gRNA is SEQ ID NO: 107—CGCUCUAUGAGUCCGAGCAGAAGAAGAA; and EMX1 xt3-gRNA is SEQ ID NO: 108—CUUGAGUUGCAAGAGUCCGAGCAGAAGAAGAA.

FIG. 15A is a representation of two gel images showing trials using EMX1.

FIG. 15B is a representation of two gel images showing trials using FANCF.

FIG. 16 is a graphical representation of in vitro cleavage of human gene HBB and top off-target site by cas9 with WT or extended guide RNA. X-1, X-2, and X-3 are extended gRNA prepared according to the method of the present invention and compared to WT sgRNA to treat sickle cell disease. ON refers to the on-target sequence and OT1 refers to an off-target sequence. Error bars represent standard deviation.

### DETAILED DESCRIPTION

In general, the present disclosure is directed to methods and systems for selection and/or design of crRNA sequences to perform gene editing using a CRISPR effector. An example aspect of the present disclosure can include selecting for crRNA sequences displaying low promiscuity while maintaining efficacy for degrading a target sequence.

For implementations of the present disclosure, the CRISPR effector can include any CRISPR effector that can be implemented as part of a CRISPR system to result in breakage of nucleotide oligomers such as RNA or DNA. Some non-limiting examples of CRISPR effectors that can be used in embodiments of the disclosure include enAsCas12a (Cas12a), RfxCas13d (Cas 13d), and/or SpyCas9 (Cas 9).

In accordance with example implementations of the present disclosure, an example embodiment can include a process for selecting crRNA sequences for CRISPR gene editing. Aspects of the process can include: identifying a target oligonucleotide sequence; transferring the target oligonucleotide sequence to a first plasmid, wherein the first plasmid further comprises an oligonucleotide sequence encoding a toxin; obtaining a second plasmid, wherein the second plasmid comprises an oligonucleotide sequence encoding a CRISPR effector (e.g., cas9); identifying a plurality of off-target oligonucleotide sequences; obtaining a plurality of third plasmids; transferring each off-target oligonucleotide sequences in a different third plasmid from the plurality of third plasmids, wherein each third plasmid from the plurality of third plasmids further comprises an oligonucleotide sequence encoding a crRNA; transfecting an organism with the first plasmid, the second plasmid, and the plurality of third plasmids, wherein, the crRNA sequence comprises an x-gRNA form, x portion including n-m nucleotides covalently linked to a gRNA portion, and the gRNA portion comprises a sequence of nucleotides complementary to a portion of the target oligonucleotide sequence.

A further example embodiment can include a system for selecting gRNA sequences for CRISPR gene editing. The example system can include: a first plasmid, where the first plasmid includes a target oligonucleotide sequence and a first oligonucleotide sequence encoding a toxin, a second plasmid, where the second plasmid comprises a second oligonucleotide sequence encoding a CRISPR effector (e.g., cas9), and a plurality of third plasmids, wherein each third plasmid of the plurality of third plasmids comprises an off-target sequence and a third oligonucleotide sequence encoding a crRNA, wherein,

The crRNA has an x-gRNA form, x portion including n-m nucleotides covalently linked to a gRNA portion, and the gRNA portion comprises a sequence of nucleotides complementary to a portion of the target oligonucleotide sequence.

As discussed, certain CRISPR effectors may display preferred recognition and/or binding to certain protospacer motifs. For instance, using a CRISPR effector of the present disclosure, the one or more protospacer motifs can include one or more from the group: TTYN, CTTV, RTTC, TATM, CTCC, TCCC, TACA, RTTS, TATA, TGTV, ANCC, CVCC, TGCC, GTCC, TTAC, or combinations thereof. In some implementations, the one or more protospacer motifs can include a subset of this group. For example, in certain embodiments, the one or more protospacer motifs are from the group: TTYN, CTTV, RTTC, TATM, CTCC, TCCC, TACA, or combinations thereof. More particularly, some embodiments can include identifying target sequences that occur downstream of the position of one or more of these protospacer motifs in the viral genome. As used herein, protospacer motifs are provided as nucleotide sequences: A—adenosine, C—cytosine, T—thymidine, G—guanosine, V—uridine, N—any nucleotide, R—adenosine or guanosine, S—guanosine or cytosine, Y—a pyrimidine (C, T, or V).

The present invention will be better understood with reference to the following non-limiting examples.

### EXAMPLES

The present examples provide aspects of embodiments of the present disclosure. These examples are not meant to limit embodiments solely to such examples herein, but rather to illustrate some possible implementations.

### Example 1

### Principle

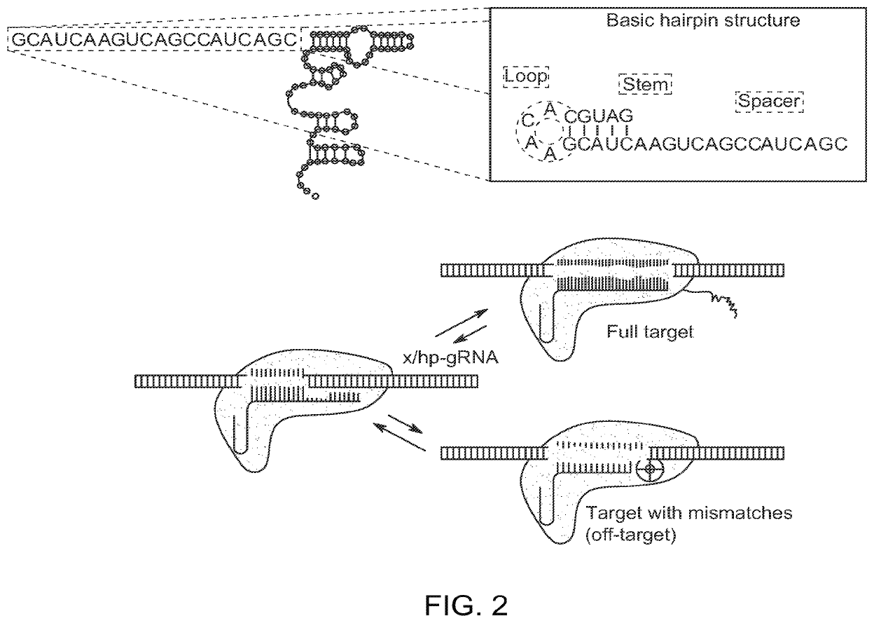

A goal of therapeutic gene editing is to be able to make any genetic or epigenetic change that may treat a genetic disorder with both high efficiency and extreme precision and specificity. Achieving extreme specificity would not only eliminate clinical risk for off-target mutational events, but would also present new therapeutic opportunities of CRISPR gene editing like allele-specific gene editing. As such, what is most exciting beyond the demonstration of feasibility for additional (specially designed) nucleotides to improve specificity is that the now available vast ‘design space’ of x-gRNA sequences creates unprecedented potential to control CRISPR effector behavior and specificity. While the targeting sequence remains fully intact, the sequences of the extra nucleotides could be made to form base-pairing secondary structures with different segments of the target-binding region with tunable strengths, which suggests that x-gRNAs could be made where the secondary structure is displaced specifically upon invasion of a ‘disease’ allele but not at the healthy allele, or generally inhibits invasion/interaction at off-targets (FIG. 2).

### Initial Results

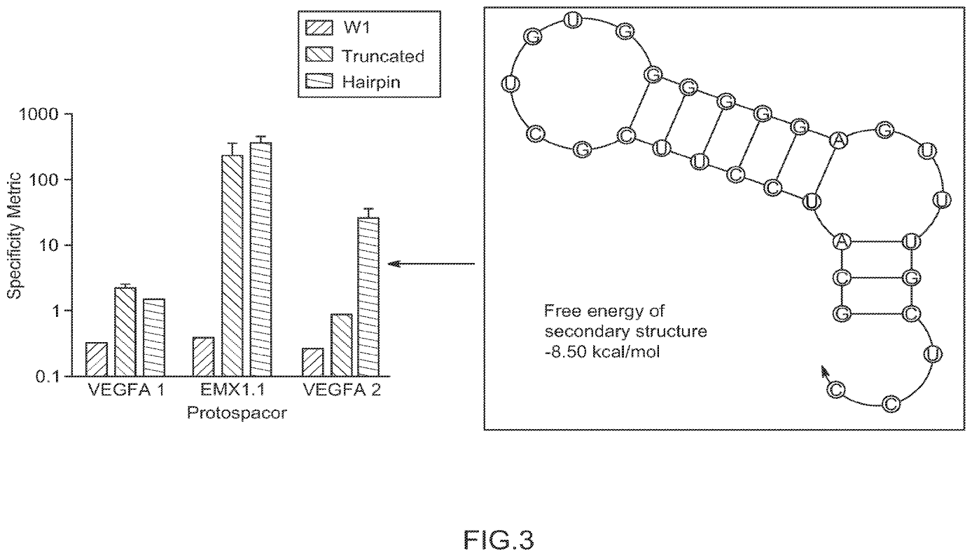

While it was found that x/hp-gRNAs could significantly outperform the state-of-the-art (FIG. 3), as other x/hp-gRNAs were manually screened, it was found some that did not make targeting more specific; others also inhibited activity on-target; and still others (controls) were not predicted to increase specificity, but which sometimes did. Ultimately, with the small sample size of x-gRNAs overarching design rules could not be defined to predict de nova increases in specificity.

### Approach

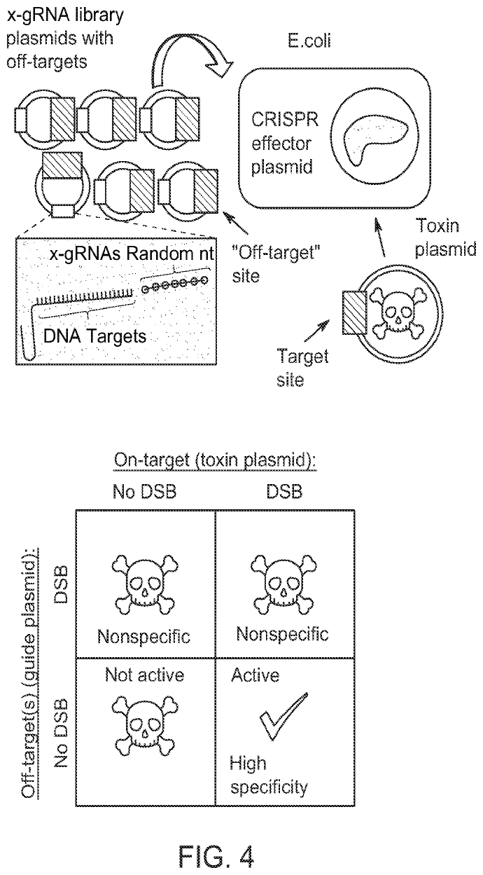

To empirically identify x-gRNAs that are both functional (mutational activity on-target) and specific (lower activity off-target or at ‘healthy’ alleles, as appropriate), a new approach was developed and validated to screen hundreds of thousands of x-gRNA variants by modifying an Escherichia coli-based selection method that has been used to isolate Cas9 and Cas12 variants with improved specificity and activity during directed evolution. The selection is based on the idea that in E. coli, cleavage or inducing DSBs in a plasmid results in plasmid degradation rather than repair, so CRISPR activity on-target can be positively selected for via degradation of a toxic plasmid that contains the targeted sequence, and CRISPR activity off-target can be negatively selected against by introducing the off-target sequences on another plasmid with antibiotic resistance genes and using antibiotics (FIG. 4).

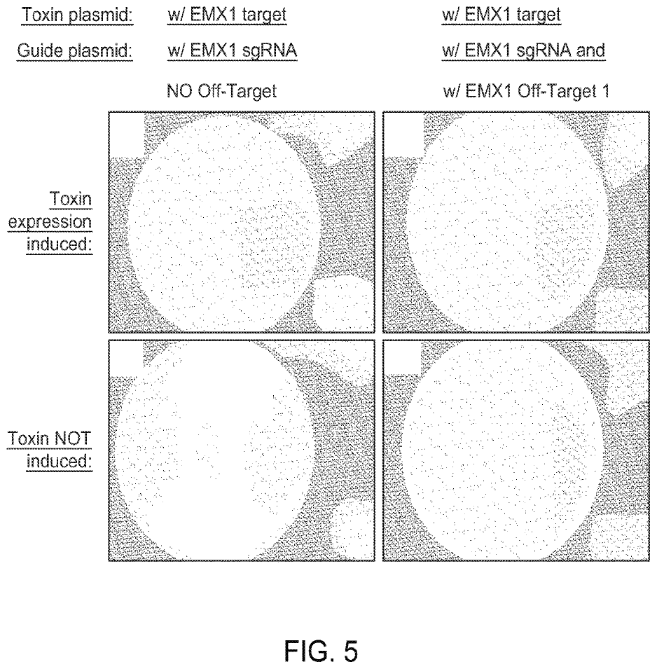

A plasmid was introduced for the tightly-controlled expression of S. pyogenes Cas9 as well as a medium copy-number plasmid for the tightly-controlled expression of E. coli toxin CcdB into which a “target’ sequence (ON) from the human EMX1 gene was cloned (FIG. 5). A third, low-copy-number plasmid expresses a well-characterized EMX1 sgRNA to the EMX1 target, but also contains a known EMX1 off-target sequence (OFF1) where Cas9 with this sgRNA exhibits significant cleavage activity (FIG. 5). After transformation and simultaneous expression of CcdB, Cas9, and the sgRNA, this screen simultaneously exhibited strong positive selection of Cas 9 activity on target (able to degrade the toxin plasmid and survive; FIG. 5, left column images) and extremely strong negative selection of off-target (unable to survive if the gRNA plasmid contained OFF1 sequence, FIG. 5, right column images). It was further confirmed this selectivity using 14 other known EMX1 off-targets (containing up to 4 mismatches on the gRNA plasmid), and in 14 out of 15 (OFF13 being the only exception) every single one exhibited significant negative selection relative to gRNA plasmids with no off-target (lacking a PAM sequence).

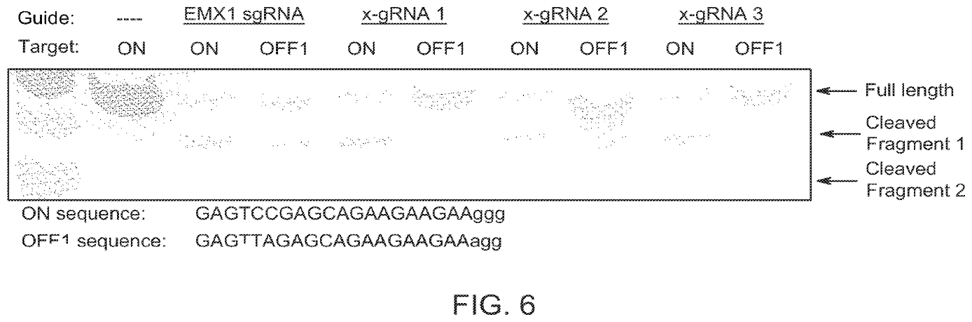

An EMX1 “x-gRNA library” was then generated to clone randomized sets of 8 nts upstream of the gRNA sequence-sets of 65,536 unique x-gRNA sequences, three different 4 nt tetraloop sequences (as previously had been done to protect the x-gRNA from degradation and promote interactions with the targeting segment), or no tetraloop. Unlike when the EMX1 sgRNA plasmid was used (FIG. 5), when the EMX1 x-gRNA library was introduced into the screening protocol, many survivors were found. Three survivor colonies were picked for Sanger sequencing of their x-gRNA, and their x-gRNAs were sequenced in vitro to test their biochemical activity using purified Cas9 at DNA with ON and OFF1 (FIG. 6). As expected, the EMX1 sgRNA exhibited cleavage activity at both ON and OFF1 in vitro, but all three selected x-gRNAs exhibited activity comparable to the sgRNA at the ON site while effectively abolishing cleavage activity at OFF1. Therefore, with no human “design” component or biophysical intuition necessary, the screen allowed de novo identification of several high-activity, high specificity x-gRNAs from a library of several hundred thousand, where most nt extensions would ordinarily be expected to either have no effect on off-target activity or be deleterious to on-target activity.

Next-generation sequencing was performed on the survivors and the top five extensions in terms of abundance were picked. Their activity was then screened in vitro at the target site and top four known off-target sites for EMX1.

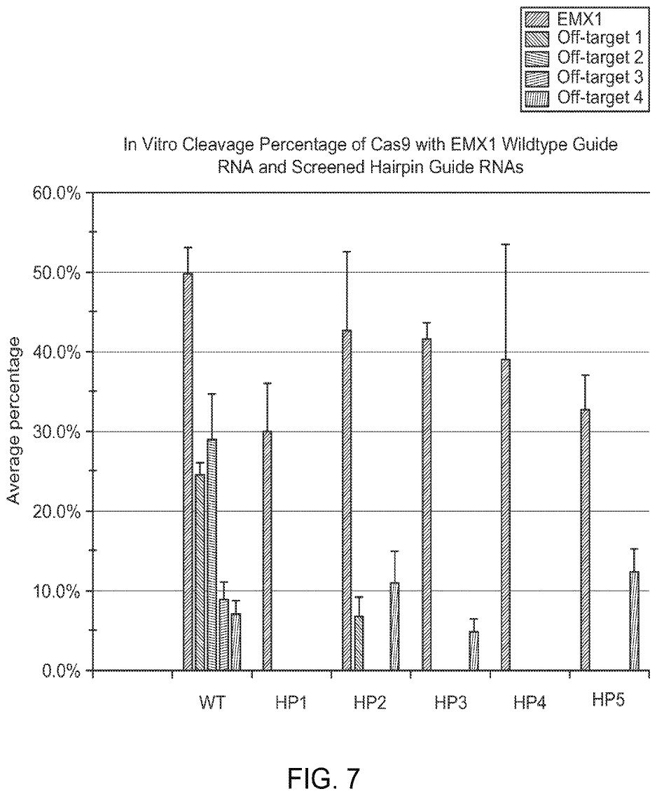

As seen in FIG. 7, these extensions allowed the Cas9 to cleave its intended target with similar efficiency as the regular gRNA, while effectively eliminating activity at all four of the top known off-targets for the EXM1 gRNA. So x-gRNAs generated through this screening approach of the present invention are broadly more specific than the regular gRNAs and therefore likely to be significantly safer for therapeutic applications. Similarly, extensions may be used with other CRISPR effectors, such as Cas12. Extensions may be on the 3′ side of the crRNA, the 5′ side of the crRNA, or combinations thereof. For example, with a Cas12 CRISPR effector, the extension may be on the 3′ side of the crRNA.

### Example 2

### Target Mutation in KRAS

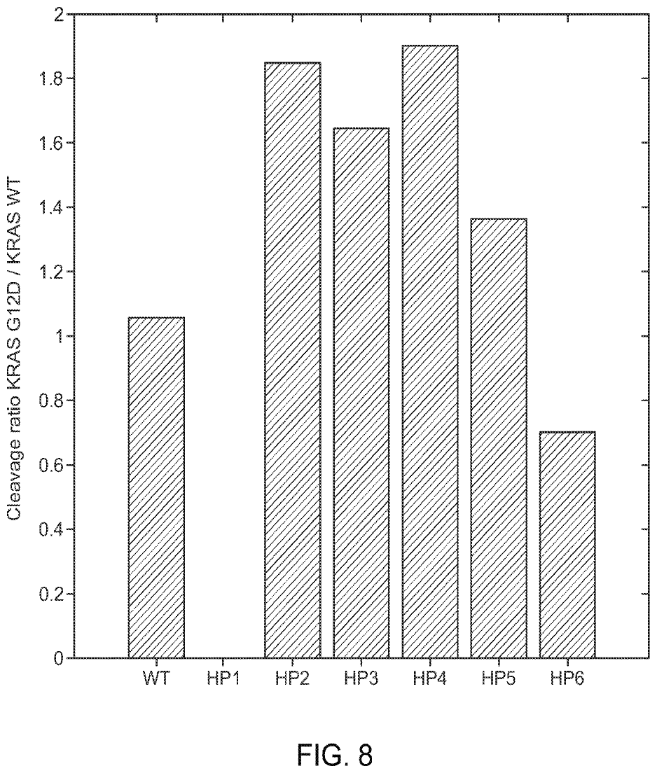

KRAS is the most highly mutated oncogene in many cancers of the lung, pancreas and colon—a driver of up to 20% of all cancer—and in many cases carries a negative prognosis. Oncogenic KRAS is considered essentially ‘undruggable’, meaning its mutationally activated and cancer-causing forms are, with one exception, unable to be inhibited by traditional pharmaceuticals. These activating mutations occur as a spectrum of SNVs at the hotspot codons 12 and 13 that have not been very easily targeted by CRISPR, or with poor specificity. To test the limits of the present invention, a screen was performed to see if Cas9 could be used to cleave DNA with a single nucleotide difference that causes cancer (KRAS G12D) while minimizing activity on the healthy sequence. Before the screen, the regular gRNA basically targeted the healthy and cancer-causing mutant sequence equally (FIG. 8). After the screen, gRNAs with hairpins shift the ratio of cleavage activity at the oncogenic mutation sequence vs healthy sequence to nearly 2:1. Cleavage efficiencies are shown in Table 1.

TABLE 1
Cleavage efficiency at cancer-causing (left) and healthy (right)
sequences for the oncogene KRAS. N = 4 independent trials.

| Cleavage Efficiency | On target (G12D) | Off-target (KRAS wt) |
|---|---|---|
| WT | 0.721676 ± 0.361 | 0.681485 ± 0.341 |
| hp1 | 0% ± 0.0 | 0% ± 0.0 |
| hp2 | 0.219905 ± 0.11 | 0.119321 ± 0.06 |
| hp3 | 0.234374 ± 0.117 | 0.142049 ± 0.071 |
| hp4 | 0.260807 ± 0.13 | 0.137071 ± 0.069 |
| hp5 | 0.55468 ± 0.277 | 0.407456 ± 0.204 |
| hp6 | 0.046955 ± 0.023 | 0.066797 ± 0.033 |

This example shows that the invention method is capable of generating gRNAs with increased “specificity” in targeting disease-causing mutations even under very difficult and stringent conditions (a single nucleotide difference).

### Example 3

### Principle

For a CRISPR guide RNA (gRNA) with a specific target but activity at known “off-target” sequences, a method was presented to screen hundreds of thousands of gRNA variants with short, randomized 5′ nucleotide extensions near its DNA-targeting segment—a modification that can increase Cas9 gene editing specificity by orders of magnitude with certain 5′-extension sequences, via some as-yet-unknown mechanism that makes de novo design of the extension sequence difficult to perform manually—to robustly identify extended gRNAs (x-gRNAs) that have been counter-selected against activity at those off-target sites and that exhibit significantly enhanced Cas9 specificity for their intended targets.

CRISPR effector Cas9 from Streptococcus pyogenes (SpyCas9) has emerged over the past several years as a powerful biotechnological tool that also holds tremendous therapeutic potential in the treatment of genetic diseases. This potential arises from the ability of CRISPR effectors to use a modular segment of their RNA co-factor, their guide RNA or ‘gRNA’, to recognize DNA sequences complementary to its ‘spacer’ segment and introduce targeted mutations into the DNA at those sites. However, oftentimes a gRNA for a specific target can cause the Cas9 nuclease to introduce “off-target” double-strand breaks (DSBs) and mutations at similar nucleotide sequences that are also present in that genome, and the possibility of unintended or uncontrolled Cas9-induced mutational events raises significant concerns for those therapeutic applications. In particular, it is becoming increasingly important to recognize that individuals may carry unique or “personal” off-target sequences for a therapeutic gRNA as a result of genetic variations that exist between people and/or across different populations, and that these unique off-target sequences must be accounted for in an era of personalized medicine.

Although there have not been any approaches developed to directly limit activity at those specifically-identified off-target sequences for a gRNA of interest, there are a few ways to reduce “off-target” activity overall and increase the specificity of CRISPR systems in general. For example, these general approaches include reducing cellular exposure to Cas9 nucleases9 or selectively inhibiting Cas9 nuclease activity altogether, as well as using engineered, “high fidelity” or “enhanced specificity” Cas9 variants such as eCas9. eCas9 effectors have amino acid substitutions designed to reduce their overall affinity for DNA in a way that decreases the probability that the effectors' latent nuclease domains will become activated at sequences with imperfect complementarity to its gRNA. These engineered Cas9 variants tend to exhibit nuclease activity that has been reduced overall by up to several orders of magnitude. Modification of the gRNA itself has also been found to modulate Cas9 specificity: gRNAs with chemically modified bases, phosphates, or sugars can exhibit increased specificity overall relative to unmodified gRNAs, although the optimal combination of modifications for a specific target/off-target can be difficult to predict de novo. Removing a few nucleotides from the 5′ end of the DNA-targeting segment of the gRNA (from 20 nt to 17 or 18 nt) to generate truncated gRNA (‘tru-gRNAs’) can also decrease off-target activity, an effect likely caused by a general destabilization of gRNA/DNA interactions when the spacers are shortened.

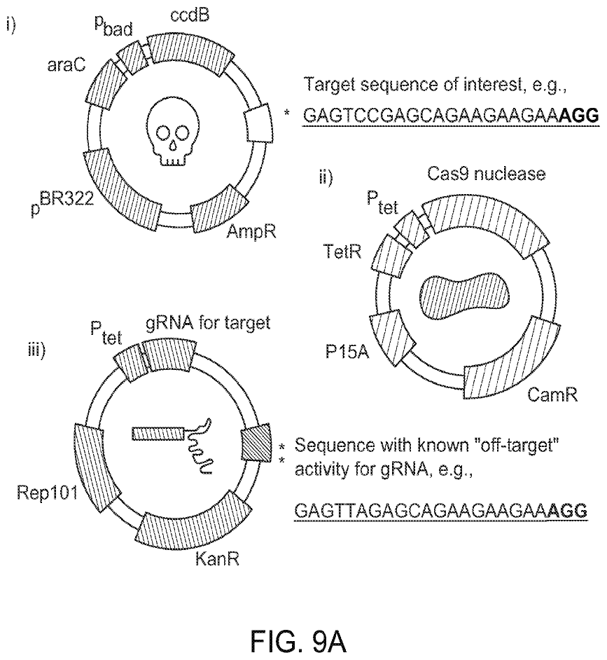

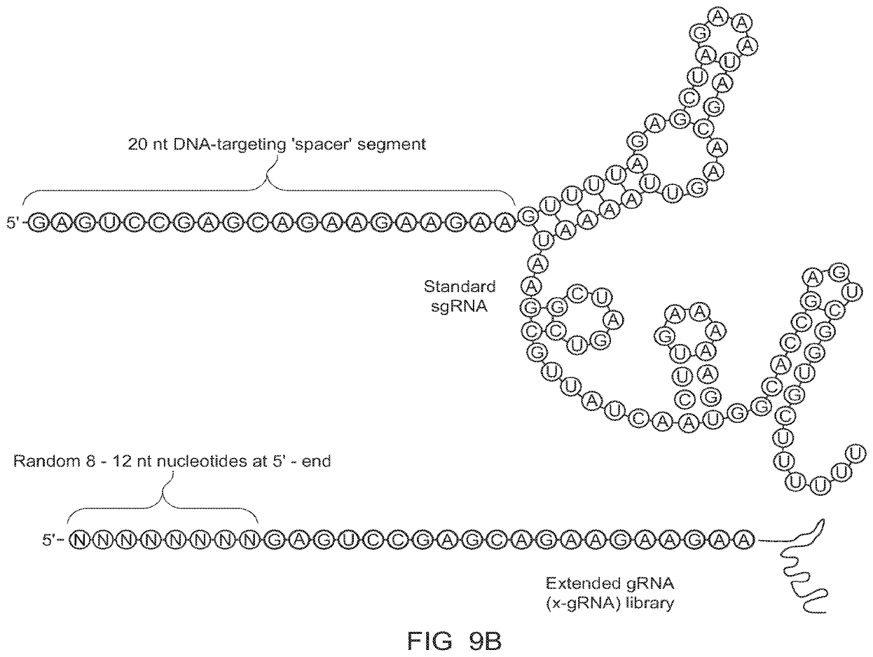

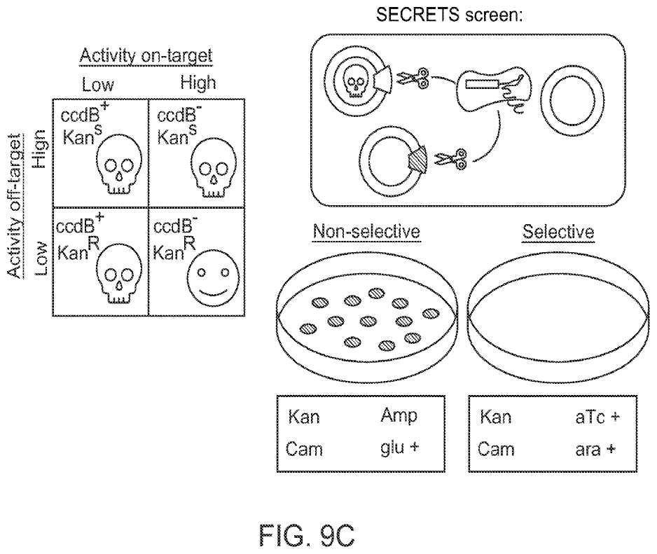

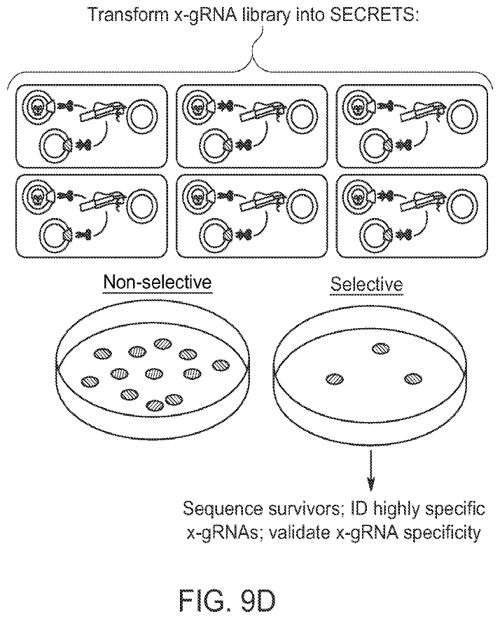

Recently, it was found that adding short nucleotide extensions (~6 to ~16 nts) to the 5′-end of the gRNA next to its DNA-targeting ‘spacer’ segment (FIG. 9B)—especially those that were predicted to form ‘hairpin’ or secondary structures with the spacer designed to interfere with gRNA interactions at specific off-target sequences-could significantly reduce Cas9 off-target activity while maintaining on-target mutational efficiencies. On average, the specificity in targeting during gene editing for these “hairpin-gRNAs” or “hp-gRNAs” increased 50-fold (and up to 200-fold) relative to gene editing using standard gRNAs, and this approach worked in diverse CRISPR effectors for multiple target sites each. While those hp-gRNAs could significantly outperform the state-of-the-art, the 5′ extended sequences were each designed and tested one-at-a-time, manually, for each targeted sequence and set of off-targets. At the time, it was also found that some of tested 5′-extensions did not effectively reduce off-target activity; others also significantly inhibited on-target activity; and still others (controls) that were not predicted to increase specificity occasionally did. Because there are a very large number of possible short 5′-extensions and because, in principle, different 5′-extensions for the same spacer sequence can be fine-tuned or optimized to limit activity vs. specific off-target sequences, the inability to predict de novo which of those sequences will increase the specificity of an associated Cas9/gRNA ribonucleoprotein (RNP) has so far limited their utility in practice in eliminating the risk of off-target mutation during gene editing.

### Materials and Methods

DNA Oligonucleotides, dsDNA, and Plasmids:

DNA sequences for all oligonucleotides, dsDNA fragments, and plasmids are listed in Tables 2, 3, and 4, respectively.

TABLE 2
Oligonucleotide and primer sequences

| Primer Name | Sequence | Assay |
|---|---|---|
| pBbA2K-RFP-Fwd (SEQ | AGTCACACTGGCTCACCTTC | Cloning (pSECRETS-A) |
| ID NO: 1) | | |
| pBbA2K-RFP-Rev (SEQ | TAACAACCCGTAAACTCGCC | Cloning (pSECRETS-A) |
| ID NO: 2) | | |
| Cas9-Fwd (SEQ ID NO: | GCACAAATAGCGTCGGATGG | Cloning (pSECRETS-A) |
| 3) | | |
| Cas9-Rev (SEQ ID NO: | GAAGGTGAGCCAGTGTGACT | Cloning (pSECRETS-A) |
| 4) | | |
| gRNA-3 (SEQ ID NO: 5) | TCGTTGTGGGAGGTGATGTC | Cloning (pSECRETS-B) |
| gRNA-5 (SEQ ID NO: 6) | GACGAAGACTCAATTG | Cloning (pSECRETS-B) |
| pBbS2k/gRNA-fwd | GACATCACCTCCCACAACGA | Cloning (pSECRETS-B) |
| (SEQ ID NO: 7) | | |
| pBbS2k/gRNA-rev (SEQ | GGTAAGGTTGGGAAGCCCTG | Cloning (pSECRETS-B) |
| ID NO: 8) | | |
| pBbS2k/gRNA-check5 | CCTGCGCCATCAGATCCTT | Cloning (pSECRETS-B) |
| (SEQ ID NO: 9) | | |
| pBbS2k/gRNA-check3 | GGTGGAGTGACGACCTTCAG | Cloning (pSECRETS-B) |
| (SEQ ID NO: 10) | | |
| pBbS2k-fwd (SEQ ID | GACATCACCTCCCACAACGA | Cloning (pSECRETS-B) |
| NO: 11) | | |
| pBbS2k-rev (SEQ ID | TAACAACCCGTAAACTCGCC | Cloning (pSECRETS-B) |
| NO: 12) | | |
| p11-Fwd (SEQ ID NO: | AAG CTT GGC TGT TTT GGC G | Cloning (pSECRETS-C) |
| 13) | | |
| p11-Rev (SEQ ID NO: | GCG TGA TAT TAC CCT GTT ATC | Cloning (pSECRETS-C) |
| 14) | CC | |
| Target-Fwd (SEQ ID | ATA ACA GGG TAA TAT CAC GC | Cloning (pSECRETS-C) |
| NO: 15) | | |
| EMX1-Rev (SEQ ID | CCG CCA AAA CAG CCA AGC | Cloning (pSECRETS-C) |
| NO: 16) | TTT TGA TGT GAT GGG AGC TCT | |
| | T | |
| FANCF-Rev (SEQ ID | CCG CCA AAA CAG CCA AGC | Cloning (pSECRETS-C) |
| NO: 17) | TTG CTC GGA AAA GCG ATC | |
| | TAG | |
| VEGFA-Rev (SEQ ID | CCG CCA AAA CAG CCA AGC | Cloning (pSECRETS-C) |
| NO: 18) | TTT CAG AAA TAG GGG GTC | |
| | CAG | |
| B-FwdUSER (SEQ ID | AGCAAG\DEOXYU\TAAAATAAG | Cloning (x-)gRNA |
| NO: 19) | GCTAGTCCG | libraries and off-targets |
| | | into pSECRETS-B |
| B-RevUSER (SEQ ID | AGTGGG\DEOXYUTCTCTAGTTA | Cloning (x-)gRNA |
| NO: 20) | GCCAGAG | libraries and off-targets |
| | | into pSECRETS-B |
| SECRETS-FwdUSER | ACCCAC\DEOXYU\GCTTACTGG | Cloning (x-)gRNA |
| (SEQ ID NO: 21) | CTTATCG | libraries and off-targets |
| | | into pSECRETS-B |
| SECRETS-RevUSER | ACTTGC\DEOXYU\ATTTCTAGCT | Cloning (x-)gRNA |
| (SEQ ID NO: 22) | CTAAAAC | libraries and off-targets |
| | | into pSECRETS-B |
| EMX1-OFF1-N8- | CCACTGCTTACTGGCTTATCGGA | Cloning EMX1 OFF1 |
| xgRNA (SEQ ID NO: | AGGGCAAGACAGATTGTCAGAG | and N8 x-gRNA library |
| 23) | TTAGAGCAGAAGAAGAAAGGCA | into pSECRETS-B |
| | TGGAGTAAAGGCAAAATTTCCC | |
| | TATCAGTGATAGAGATTGACAT | |
| | CCCTATCAGTGATAGAGATACT | |
| | GAGCACNNNNNNNNGAGTCCGA | |
| | GCAGAAGAAGAAGTTTTAGAGC | |
| | TAGAAATAGCAAGTTAAA | |
| EMX1-OFF1(noPAM)- | CCACTGCTTACTGGCTTATCGGA | Cloning EMX1 (no |
| N8-xgRNA (SEQ ID | AGGGCAAGACAGATTGTCAGAG | OFF1) N8 x-gRNA |
| NO: 24) | TTAGAGCAGAAGAAGAATTTCA | library into pSECRETS- |
| | TGGAGTAAAGGCAAAATTTCCC | B |
| | TATCAGTGATAGAGATTGACAT | |
| | CCCTATCAGTGATAGAGATACT | |
| | GAGCACNNNNNNNNGAGTCCGA | |
| | GCAGAAGAAGAAGTTTTAGAGC | |
| | TAGAAATAGCAAGTTAAA | |
| FANCF-OFF1-N8- | CCACTGCTTACTGGCTTATCGGA | Cloning FANCF OFF1 |
| xgRNA (SEQ ID NO: | AGGGATCGTCCTGACCCCGGGA | and N8 x-gRNA library |
| 25) | ACCCCGTCTGCAGCACCAGGCC | into pSECRETS-B |
| | CCCTCCGTGGAGAAAATTTCCCT | |
| | ATCAGTGATAGAGATTGACATC | |
| | CCTATCAGTGATAGAGATACTG | |
| | AGCACNNNNNNNNGGAATCCCT | |
| | TCTGCAGCACCGTTTTAGAGCTA | |
| | GAAATAGCAAG | |
| FANCF-OFF1(noPAM)- | CCACTGCTTACTGGCTTATCGGA | Cloning FANCF (no |
| N8-xgRNA (SEQ ID | AGGGCTTCGCGCACCTCATGGA | OFF1) N8 x-gRNA |
| NO: 26) | ATCCCTTCTGCAGCACCTTTATC | library into pSECRETS- |
| | GCTTTTCCGAGCAAATTTCCCTA | B |
| | TCAGTGATAGAGATTGACATCC | |
| | CTATCAGTGATAGAGATACTGA | |
| | GCACNNNNNNNNGGAATCCCTT | |
| | CTGCAGCACCGTTTTAGAGCTA | |
| | GAAATAGCAAG | |
| VEGFA-OFF1-N8- | CCACTGCTTACTGGCTTATCGGA | Cloning VEGFA OFF1 |
| xgRNA (SEQ ID NO: | AGGGAAGGACGGATTTGTGGGA | and N8 x-gRNA library |
| 27) | TGGAGGGAGTTTGCTCCTGGGG | into pSECRETS-B |
| | TGTCAGAATGTCCAAATTTCCCT | |
| | ATCAGTGATAGAGATTGACATC | |
| | CCTATCAGTGATAGAGATACTG | |
| | AGCACNNNNNNNNGGGTGGGG | |
| | GGAGTTTGCTCCGTTTTAGAGCT | |
| | AGAAATAGCAAG | |
| VEGFA-OFF1(noPAM)- | CCACTGCTTACTGGCTTATCGGA | Cloning VEGFA (no |
| N8-xgRNA (SEQ ID | AGGGTGGGCTTTGGAAAGGGGG | OFF1) N8 x-gRNA |
| NO: 28) | TGGGGGGAGTTTGCTCCTTTACC | library into pSECRETS- |
| | CCCTATTTCTGAAAATTTCCCTA | B |
| | TCAGTGATAGAGATTGACATCC | |
| | CTATCAGTGATAGAGATACTGA | |
| | GCACNNNNNNNNGGGTGGGGG | |
| | GAGTTTGCTCCGTTTTAGAGCTA | |
| | GAAATAGCAAG | |
| SECRETS-BSeq51 | ACACTCTTTCCCTACACGACGCT | Sequencing pSECRETS- |
| (SEQ ID NO: 29) | CTTCCGATCT TTTT | B (x-)gRNA libraries |
| | GAGCGGATACATATTTGAATG | |
| SECRETS-BSeq52 | ACACTCTTTCCCTACACGACGCT | Sequencing pSECRETS- |
| (SEQ ID NO: 30) | CTTCCGATCT AAAA A | B (x-)gRNA libraries |
| | GAGCGGATACATATTTGAATG | |
| SECRETS-BSeq53 (SEQ | ACACTCTTTCCCTACACGACGCT | Sequencing pSECRETS- |
| ID NO: 31) | CTTCCGATCT GGGG TA | B (x-)gRNA libraries |
| | GAGCGGATACATATTTGAATG | |
| SECRETS-BSeq54 | ACACTCTTTCCCTACACGACGCT | Sequencing pSECRETS- |
| (SEQ ID NO: 32) | CTTCCGATCT CCCC GCC | B (x-)gRNA libraries |
| | GAGCGGATACATATTTGAATG | |
| SECRETS-BSeq31 | GACTGGAGTTCAGACGTGTGCT | Sequencing pSECRETS- |
| (SEQ ID NO: 33) | CTTCCGATCT TTTT | B (x-)gRNA libraries |
| | AAGTTGATAACGGACTAGCC | |
| SECRETS-BSeq32 (SEQ | GACTGGAGTTCAGACGTGTGCT | Sequencing pSECRETS- |
| ID NO: 34) | CTTCCGATCT AAAA A | B (x-)gRNA libraries |
| | AAGTTGATAACGGACTAGCC | |
| SECRETS-BSeq33 (SEQ | GACTGGAGTTCAGACGTGTGCT | Sequencing pSECRETS- |
| ID NO: 35) | CTTCCGATCT GGGG TA | B (x-)gRNA libraries |
| | AAGTTGATAACGGACTAGCC | |
| SECRETS-BSeq34 (SEQ | GACTGGAGTTCAGACGTGTGCT | Sequencing pSECRETS- |
| ID NO: 36) | CTTCCGATCT CCCC GCC | B (x-)gRNA libraries |
| | AAGTTGATAACGGACTAGCC | |
| EMX1-Fwd (SEQ ID | TATGTAGCCTCAGTCTTCCCATC | T7E1 mutation detection |
| NO: 37) | A | |
| EMX1-Rev (SEQ ID | TGGTTGCCCACCCTAGTCATTG | T7E1 mutation detection |
| NO: 38) | | |
| EMX1-OT1-Fwd (SEQ | GAAAACTCAAAGAAATGCCCAA | T7E1 mutation detection |
| ID NO: 39) | TCA | |
| EMX1-OT1-Rev (SEQ | TCTGTGGAGGCAATAAAGTGAA | T7E1 mutation detection |
| ID NO: 40) | ATAA | |
| FANCF-Fwd (SEQ ID | GGGCCTGGAAGTTCGCTAAT | T7E1 mutation detection |
| NO: 41) | | |
| FANCF-Rev (SEQ ID | AGGCGTATCATTTCGCGGAT | T7E1 mutation detection |
| NO: 42) | | |
| FANCF-OT1-Fwd (SEQ | GTTGCGTTTGAGGATGAGCA | T7E1 mutation detection |
| ID NO: 43) | | |
| FANCF-OT1-Rev (SEQ | GAAATCTCCCACGAGTCTCCG | T7E1 mutation detection |
| ID NO: 44) | | |
| VEGFA-Fwd (SEQ ID | TCCAGATGGCACATTGTCAG | T7E1 mutation detection |
| NO: 45) | | |
| VEGFA-Rev (SEQ ID | AGGGAGCAGGAAAGTGAGGT | T7E1 mutation detection |
| NO: 46) | | |
| VEGFA-OT1-Fwd (SEQ | ACCCCACAGCCAGGTTTTCA | T7E1 mutation detection |
| ID NO: 47) | | |
| VEGFA-OT1-Rev (SEQ | GAATCACTGCACCTGGCCATC | T7E1 mutation detection |
| ID NO: 48) | | |
| EMX1 On fwd 1 (SEQ | ACACTCTTTCCCTACACGACGCT | Illumina Sequencing |
| ID NO: 49) | CTTCCGATCTATCACGAGTTTCT | |
| | CATCTGTGCCCCT | |
| EMX1 On fwd 2 (SEQ | ACACTCTTTCCCTACACGACGCT | Illumina Sequencing |
| ID NO: 50) | CTTCCGATCTCGATGTAAGTTTC | |
| | TCATCTGTGCCCCT | |
| EMX1 On fwd 3 (SEQ | ACACTCTTTCCCTACACGACGCT | Illumina Sequencing |
| ID NO: 51) | CTTCCGATCTTTAGGCTAAGTTT | |
| | CTCATCTGTGCCCCT | |
| EMX1 On fwd 4 (SEQ | ACACTCTTTCCCTACACGACGCT | Illumina Sequencing |
| ID NO: 52) | CTTCCGATCTTGACCAGTAAGTT | |
| | TCTCATCTGTGCCCCT | |
| EMX1 On fwd 5 (SEQ | ACACTCTTTCCCTACACGACGCT | Illumina Sequencing |
| ID NO: 53) | CTTCCGATCT ACATGT CGTA | |
| | AGTTTCTCATCTGTGCCCCT | |
| EMX1 On fwd 6 (SEQ | ACACTCTTTCCCTACACGACGCT | Illumina Sequencing |
| ID NO: 54) | CTTCCGATCT GCCAAT ACGTA | |
| | AGTTTCTCATCTGTGCCCCT | |
| EMX1 On rev 1 (SEQ ID | GACTGGAGTTCAGACGTGTGCT | Illumina Sequencing |
| NO: 55) | CTTCCGATCT CAGATC | |
| | ATCGATGTCCTCCCCATTGG | |
| EMX1 On rev 2 (SEQ ID | GACTGGAGTTCAGACGTGTGCT | Illumina Sequencing |
| NO: 56) | CTTCCGATCT ACTTGA | |
| | ATCGATGTCCTCCCCATTGG | |
| EMX1 On rev 3 (SEQ ID | GACTGGAGTTCAGACGTGTGCT | Illumina Sequencing |
| NO: 57) | CTTCCGATCT GATCAG | |
| | ATCGATGTCCTCCCCATTGG | |
| EMX1 On rev 4 (SEQ ID | GACTGGAGTTCAGACGTGTGCT | Illumina Sequencing |
| NO: 58) | CTTCCGATCT TAGCTT | |
| | ATCGATGTCCTCCCCATTGG | |
| EMX1 On rev 5 (SEQ ID | GACTGGAGTTCAGACGTGTGCT | Illumina Sequencing |
| NO: 59) | CTTCCGATCT GGCTAG | |
| | ATCGATGTCCTCCCCATTGG | |
| EMX1 On rev 6 (SEQ ID | GACTGGAGTTCAGACGTGTGCT | Illumina Sequencing |
| NO: 60) | CTTCCGATCT CTTGTA | |
| | ATCGATGTCCTCCCCATTGG | |
| EMX1 OT1 fwd 1 (SEQ | ACACTCTTTCCCTACACGACGCT | Illumina Sequencing |
| ID NO: 61) | CTTCCGATCT ATCACG | |
| | CGCTTGTCCATGTCTAGGAA | |
| EMX1 OT1 fwd 2 (SEQ | ACACTCTTTCCCTACACGACGCT | Illumina Sequencing |
| ID NO: 62) | CTTCCGATCT CGATGT A | |
| | CGCTTGTCCATGTCTAGGAA | |
| EMX1 OT1 fwd 3 (SEQ | ACACTCTTTCCCTACACGACGCT | Illumina Sequencing |
| ID NO: 63) | CTTCCGATCT TTAGGC TA | |
| | CGCTTGTCCATGTCTAGGAA | |
| EMX1 OT1 fwd 4 (SEQ | ACACTCTTTCCCTACACGACGCT | Illumina Sequencing |
| ID NO: 64) | CTTCCGATCT TGACCA GTA | |
| | CGCTTGTCCATGTCTAGGAA | |
| EMX1 OT1 fwd 5 (SEQ | ACACTCTTTCCCTACACGACGCT | Illumina Sequencing |
| ID NO: 65) | CTTCCGATCT ACATGT CGTA | |
| | CGCTTGTCCATGTCTAGGAA | |
| EMX1 OT1 fwd 6 (SEQ | ACACTCTTTCCCTACACGACGCT | Illumina Sequencing |
| ID NO: 66) | CTTCCGATCT GCCAAT ACGTA | |
| | CGCTTGTCCATGTCTAGGAA | |
| EMX1 OT1 rev 1 (SEQ | GACTGGAGTTCAGACGTGTGCT | Illumina Sequencing |
| ID NO: 67) | CTTCCGATCT CAGATC | |
| | TGGCATGGCAAGACAGATTG | |
| EMX1 OT1 rev 2 (SEQ | GACTGGAGTTCAGACGTGTGCT | Illumina Sequencing |
| ID NO: 68) | CTTCCGATCT ACTTGA | |
| | TGGCATGGCAAGACAGATTG | |
| EMX1 OT1 rev 3 (SEQ | GACTGGAGTTCAGACGTGTGCT | Illumina Sequencing |
| ID NO: 69) | CTTCCGATCT GATCAG | |
| | TGGCATGGCAAGACAGATTG | |
| EMX1 OT1 rev 4 (SEQ | GACTGGAGTTCAGACGTGTGCT | Illumina Sequencing |
| ID NO: 70) | CTTCCGATCT TAGCTT | |
| | TGGCATGGCAAGACAGATTG | |
| EMX1 OT1 rev 5 (SEQ | GACTGGAGTTCAGACGTGTGCT | Illumina Sequencing |
| ID NO: 71) | CTTCCGATCT GGCTAG | |
| | TGGCATGGCAAGACAGATTG | |
| EMX1 OT1 rev 6 (SEQ | GACTGGAGTTCAGACGTGTGCT | Illumina Sequencing |
| ID NO: 72) | CTTCCGATCT CTTGTA | |
| | TGGCATGGCAAGACAGATTG | |
| FANCF On fwd 1 (SEQ | ACACTCTTTCCCTACACGACGCT | Illumina Sequencing |
| ID NO: 73) | CTTCCGATCT ATCACG | |
| | CTCCAGAGCCGTGCGAATG | |
| FANCF On fwd 2 (SEQ | ACACTCTTTCCCTACACGACGCT | Illumina Sequencing |
| ID NO: 74) | CTTCCGATCT CGATGT A | |
| | CTCCAGAGCCGTGCGAATG | |
| FANCF On fwd 3 (SEQ | ACACTCTTTCCCTACACGACGCT | Illumina Sequencing |
| ID NO: 75) | CTTCCGATCT TTAGGC TA | |
| | CTCCAGAGCCGTGCGAATG | |
| FANCF On fwd 4 (SEQ | ACACTCTTTCCCTACACGACGCT | Illumina Sequencing |
| ID NO: 76) | CTTCCGATCT TGACCA GTA | |
| | CTCCAGAGCCGTGCGAATG | |
| FANCF On fwd 5 (SEQ | ACACTCTTTCCCTACACGACGCT | Illumina Sequencing |
| ID NO: 77) | CTTCCGATCT ACATGT CGTA | |
| | CTCCAGAGCCGTGCGAATG | |
| FANCF On fwd 6 (SEQ | ACACTCTTTCCCTACACGACGCT | Illumina Sequencing |
| ID NO: 78) | CTTCCGATCT GCCAAT ACGTA | |
| | CTCCAGAGCCGTGCGAATG | |
| FANCF On rev 1 (SEQ | GACTGGAGTTCAGACGTGTGCT | Illumina Sequencing |
| ID NO: 79) | CTTCCGATCT CAGATC | |
| | AATCAGTACGCAGAGAGTCGC | |
| FANCF On rev 2 (SEQ | GACTGGAGTTCAGACGTGTGCT | Illumina Sequencing |
| ID NO: 80) | CTTCCGATCT ACTTGA | |
| | AATCAGTACGCAGAGAGTCGC | |
| FANCF On rev 3 (SEQ | GACTGGAGTTCAGACGTGTGCT | Illumina Sequencing |
| ID NO: 81) | CTTCCGATCT GATCAG | |
| | AATCAGTACGCAGAGAGTCGC | |
| FANCF On rev 4 (SEQ | GACTGGAGTTCAGACGTGTGCT | Illumina Sequencing |
| ID NO: 82) | CTTCCGATCT TAGCTT | |
| | AATCAGTACGCAGAGAGTCGC | |
| FANCF On rev 5 (SEQ | GACTGGAGTTCAGACGTGTGCT | Illumina Sequencing |
| ID NO: 83) | CTTCCGATCT GGCTAG | |
| | AATCAGTACGCAGAGAGTCGC | |
| FANCF On rev 6 (SEQ | GACTGGAGTTCAGACGTGTGCT | Illumina Sequencing |
| ID NO: 84) | CTTCCGATCT CTTGTA | |
| | AATCAGTACGCAGAGAGTCGC | |
| FANCF OT1 fwd 1 | ACACTCTTTCCCTACACGACGCT | Illumina Sequencing |
| (SEQ ID NO: 85) | CTTCCGATCT ATCACG | |
| | AGTTGGTGTCAATACAACTCCCC | |
| | T | |
| FANCF OT1 fwd 2 | ACACTCTTTCCCTACACGACGCT | Illumina Sequencing |
| (SEQ ID NO: 86) | CTTCCGATCT CGATGT A | |
| | AGTTGGTGTCAATACAACTCCCC | |
| | T | |
| FANCF OT1 fwd 3 | ACACTCTTTCCCTACACGACGCT | Illumina Sequencing |
| (SEQ ID NO: 87) | CTTCCGATCT TTAGGC TA | |
| | AGTTGGTGTCAATACAACTCCCC | |
| | T | |
| FANCF OTI fwd 4 | ACACTCTTTCCCTACACGACGCT | Illumina Sequencing |
| (SEQ ID NO: 88) | CTTCCGATCT TGACCA GTA | |
| | AGTTGGTGTCAATACAACTCCCC | |
| | T | |
| FANCF OT1 fwd 5 | ACACTCTTTCCCTACACGACGCT | Illumina Sequencing |
| (SEQ ID NO: 89) | CTTCCGATCT ACATGT CGTA | |
| | AGTTGGTGTCAATACAACTCCCC | |
| | T | |
| FANCF OT1 fwd 6 | ACACTCTTTCCCTACACGACGCT | Illumina Sequencing |
| (SEQ ID NO: 90) | CTTCCGATCT GCCAAT ACGTA | |
| | AGTTGGTGTCAATACAACTCCCC | |
| | T | |
| FANCF OT1 rev 1 (SEQ | GACTGGAGTTCAGACGTGTGCT | Illumina Sequencing |
| ID NO: 91) | CTTCCGATCT CAGATC | |
| | CGCCAGCACTTTCTAAGGAATC | |
| FANCF OT1 rev 2 (SEQ | GACTGGAGTTCAGACGTGTGCT | Illumina Sequencing |
| ID NO: 92) | CTTCCGATCT ACTTGA | |
| | CGCCAGCACTTTCTAAGGAATC | |
| FANCF OT1 rev 3 (SEQ | GACTGGAGTTCAGACGTGTGCT | Illumina Sequencing |
| ID NO: 93) | CTTCCGATCT GATCAG | |
| | CGCCAGCACTTTCTAAGGAATC | |
| FANCF OT1 rev 4 (SEQ | GACTGGAGTTCAGACGTGTGCT | Illumina Sequencing |
| ID NO: 94) | CTTCCGATCT TAGCTT | |
| | CGCCAGCACTTTCTAAGGAATC | |
| FANCF OT1 rev 5 (SEQ | GACTGGAGTTCAGACGTGTGCT | Illumina Sequencing |
| ID NO: 95) | CTTCCGATCT GGCTAG | |
| | CGCCAGCACTTTCTAAGGAATC | |
| FANCF OT1 rev 6 (SEQ | GACTGGAGTTCAGACGTGTGCT | Illumina Sequencing |
| ID NO: 96) | CTTCCGATCT CTTGTA | |
| | CGCCAGCACTTTCTAAGGAATC | |
| pSECRETS-A | GGCGAGTTTACGGGTTGTTAAACCTTCGATT | TetR-pAmp-pltetO1- |
| gBlock (SEQ | CCGACCTCATTAAGCAGCTCTAATGCGCTGT | Cas9 for pBbA2c |
| ID NO: 97) | TAATCACTTTACTTTTATCTAAACGAGACAT | |
| | ACTCTTCCTTTTTCAATATTATTGAAGCATTT | |
| | ATCAGGGTTATTGTCTCATGAGCGGATACAT | |
| | ATTTGAATGTATTTAGAAAAATAAACAAATA | |
| | GGGGTTCCGCGCACATTTCCCCGAAAAGTGC | |
| | CACCTGACGTCCTCTGGCTAACTAGAGAACC | |
| | CACTGCTTACTGGCTTATCGAAATTTCCCTA | |
| | TCAGTGATAGAGATTGACATCCCTATCAGTG | |
| | ATAGAGATACTGAGCACATCAGCAGGACGC | |
| | ACTGACCAGGGAGACCCAAGCTTGCCACCA | |
| | TGGTGTACCCCTACGACGTGCCCGACTACGC | |
| | CGAATTGCCTCCAAAAAAGAAGAGAAAGGT | |
| | AGGGATCCGAACCATGGATAAGAAATACTC | |
| | AATAGGCTTAGATATCGGCACAAATAGCGT | |
| | CGGATGG | |
| pSECRETS-B | GGCGAGTTTACGGGTTGTTAAACCTTCGATT | TetR-pAmp-pltetO1- |
| gBlock (SEQ | CCGACCTCATTAAGCAGCTCTAATGCGCTGT | gRNA (BsaI cassette) |
| ID NO: 98) | TAATCACTTTACTTTTATCTAAACGAGACAT | |
| | ACTCTTCCTTTTTCAATATTATTGAAGCATTT | |
| | ATCAGGGTTATTGTCTCATGAGCGGATACAT | |
| | ATTTGAATGTATTTAGAAAAATAAACAAATA | |
| | GGGGTTCCGCGCACATTTCCCCGAAAAGTGC | |
| | CACCTGACGTCCTCTGGCTAACTAGAGAACC | |
| | CACTGCTTACTGGCTTATCGAAATTTCCCTA | |
| | TCAGTGATAGAGATTGACATCCCTATCAGTG | |
| | ATAGAGATACTGAGCACGTGAGACCCATGC | |
| | CATAGCGTTGTTTAGGGATAACAGGGTAATA | |
| | CTGTCCACACAATCTGCCCTGGTCTCCGTTTT | |
| | AGAGCTAGAAATAGCAAGTTAAAATAAGGC | |
| | TAGTCCGTTATCAACTTGAAAAAGTGGCACC | |
| | GAGTCGGTGCTTTTTTGGCCGGCATGGTCCC | |
| | AGCCTCCTCGCTGGCGCCGGCTGGGCAACAT | |
| | GCTTCGGCATGGCGAATGGGACGACATCAC | |
| | CTCCCACAACGA | |

TABLE 3
dsDNA fragments used to clone pSECRETS plasmids

| | | |
|---|---|---|
| pSECRETS-A | GGCGAGTTTACGGGTTGTTAAACCTTCGATT | TetR-pAmp-pltetO1- |
| gBlock (SEQ | CCGACCTCATTAAGCAGCTCTAATGCGCTGT | Cas9 for pBbA2c |
| ID NO: 97) | TAATCACTTTACTTTTATCTAAACGAGACAT | |
| | ACTCTTCCTTTTTCAATATTATTGAAGCATTT | |
| | ATCAGGGTTATTGTCTCATGAGCGGATACAT | |
| | ATTTGAATGTATTTAGAAAAATAAACAAATA | |
| | GGGGTTCCGCGCACATTTCCCCGAAAAGTGC | |
| | CACCTGACGTCCTCTGGCTAACTAGAGAACC | |
| | CACTGCTTACTGGCTTATCGAAATTTCCCTA | |
| | TCAGTGATAGAGATTGACATCCCTATCAGTG | |
| | ATAGAGATACTGAGCACATCAGCAGGACGC | |
| | ACTGACCAGGGAGACCCAAGCTTGCCACCA | |
| | TGGTGTACCCCTACGACGTGCCCGACTACGC | |
| | CGAATTGCCTCCAAAAAAGAAGAGAAAGGT | |
| | AGGGATCCGAACCATGGATAAGAAATACTC | |
| | AATAGGCTTAGATATCGGCACAAATAGCGT | |
| | CGGATGG | |
| pSECRETS-B | GGCGAGTTTACGGGTTGTTAAACCTTCGATT | TetR-pAmp-pltetO1- |
| gBlock (SEQ | CCGACCTCATTAAGCAGCTCTAATGCGCTGT | gRNA (BsaI cassette) |
| ID NO: 98) | TAATCACTTTACTTTTATCTAAACGAGACAT | |
| | ACTCTTCCTTTTTCAATATTATTGAAGCATTT | |
| | ATCAGGGTTATTGTCTCATGAGCGGATACAT | |
| | ATTTGAATGTATTTAGAAAAATAAACAAATA | |
| | GGGGTTCCGCGCACATTTCCCCGAAAAGTGC | |
| | CACCTGACGTCCTCTGGCTAACTAGAGAACC | |
| | CACTGCTTACTGGCTTATCGAAATTTCCCTA | |
| | TCAGTGATAGAGATTGACATCCCTATCAGTG | |
| | ATAGAGATACTGAGCACGTGAGACCCATGC | |
| | CATAGCGTTGTTTAGGGATAACAGGGTAATA | |
| | CTGTCCACACAATCTGCCCTGGTCTCCGTTTT | |
| | AGAGCTAGAAATAGCAAGTTAAAATAAGGC | |
| | TAGTCCGTTATCAACTTGAAAAAGTGGCACC | |
| | GAGTCGGTGCTTTTTTGGCCGGCATGGTCCC | |
| | AGCCTCCTCGCTGGCGCCGGCTGGGCAACAT | |
| | GCTTCGGCATGGCGAATGGGACGACATCAC | |
| | CTCCCACAACGA | |

TABLE 4
Plasmid Sequences

pSECRETS-A (SEQ ID NO: 99):
GACGTCTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATAGCT
TGTCGTAATAATGGCGGCATACTATCAGTAGTAGGTGTTTCCCTTTCTTCTTTAGCGA
CTTGATGCTCTTGATCTTCCAATACGCAACCTAAAGTAAAATGCCCCACAGCGCTGA
GTGCATATAATGCATTCTCTAGTGAAAAACCTTGTTGGCATAAAAAGGCTAATTGAT
TTTCGAGAGTTTCATACTGTTTTTCTGTAGGCCGTGTACCTAAATGTACTTTTGCTCC
ATCGCGATGACTTAGTAAAGCACATCTAAAACTTTTAGCGTTATTACGTAAAAAATC
TTGCCAGCTTTCCCCTTCTAAAGGGCAAAAGTGAGTATGGTGCCTATCTAACATCTC
AATGGCTAAGGCGTCGAGCAAAGCCCGCTTATTTTTTACATGCCAATACAATGTAGG
CTGCTCTACACCTAGCTTCTGGGCGAGTTTACGGGTTGTTAAACCTTCGATTCCGACC
TCATTAAGCAGCTCTAATGCGCTGTTAATCACTTTACTTTTATCTAAACGAGACATAC
TCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCG
AAAAGTGCCACCTGACGTCCTCTGGCTAACTAGAGAACCCACTGCTTACTGGCTTAT
CGAAATTTCCCTATCAGTGATAGAGATTGACATCCCTATCAGTGATAGAGATACTGA
GCACATCAGCAGGACGCACTGACCAGGGAGACCCAAGCTTGCCACCATGGTGTACC
CCTACGACGTGCCCGACTACGCCGAATTGCCTCCAAAAAAGAAGAGAAAGGTAGGG
ATCCGAACCATGGATAAGAAATACTCAATAGGCTTAGATATCGGCACAAATAGCGT
CGGATGGGCGGTGATCACTGATGAATATAAGGTTCCGTCTAAAAAGTTCAAGGTTCT
GGGAAATACAGACCGCCACAGTATCAAAAAAAATCTTATAGGGGCTCTTTTATTTGA
CAGTGGAGAGACAGCGGAAGCGACTCGTCTCAAACGGACAGCTCGTAGAAGGTATA
CACGTCGGAAGAATCGTATTTGTTATCTACAGGAGATTTTTTCAAATGAGATGGCGA
AAGTAGATGATAGTTTCTTTCATCGACTTGAAGAGTCTTTTTTGGTGGAAGAAGACA
AGAAGCATGAACGTCATCCTATTTTTGGAAATATAGTAGATGAAGTTGCTTATCATG
AGAAATATCCAACTATCTATCATCTGCGAAAAAAATTGGTAGATTCTACTGATAAAG
CGGATTTGCGCTTAATCTATTTGGCCTTAGCGCATATGATTAAGTTTCGTGGTCATTT
TTTGATTGAGGGAGATTTAAATCCTGATAATAGTGATGTGGACAAACTATTTATCCA
GTTGGTACAAACCTACAATCAATTATTTGAAGAAAACCCTATTAACGCAAGTGGAGT
AGATGCTAAAGCGATTCTTTCTGCACGATTGAGTAAATCAAGACGATTAGAAAATCT
CATTGCTCAGCTCCCCGGTGAGAAGAAAAATGGCTTATTTGGGAATCTCATTGCTTT
GTCATTGGGTTTGACCCCTAATTTTAAATCAAATTTTGATTTGGCAGAAGATGCTAA
ATTACAGCTTTCAAAAGATACTTACGATGATGATTTAGATAATTTATTGGCGCAAAT
TGGAGATCAATATGCTGATTTGTTTTTGGCAGCTAAGAATTTATCAGATGCTATTTTA
CTTTCAGATATCCTAAGAGTAAATACTGAAATAACTAAGGCTCCCCTATCAGCTTCA
ATGATTAAACGCTACGATGAACATCATCAAGACTTGACTCTTTTAAAAGCTTTAGTT
CGACAACAACTTCCAGAAAAGTATAAAGAAATCTTTTTTGATCAATCAAAAAACGG
ATATGCAGGTTATATTGATGGGGGAGCTAGCCAAGAAGAATTTTATAAATTTATCAA
ACCAATTTTAGAAAAAATGGATGGTACTGAGGAATTATTGGTGAAACTAAATCGTG
AAGATTTGCTGCGCAAGCAACGGACCTTTGACAACGGCTCTATTCCCCATCAAATTC
ACTTGGGTGAGCTGCATGCTATTTTGAGAAGACAAGAAGACTTTTATCCATTTTTAA
AAGACAATCGTGAGAAGATTGAAAAAATCTTGACTTTTCGAATTCCTTATTATGTTG
GTCCATTGGCGCGTGGCAATAGTCGTTTTGCATGGATGACTCGGAAGTCTGAAGAAA
CAATTACCCCATGGAATTTTGAAGAAGTTGTCGATAAAGGTGCTTCAGCTCAATCAT
TTATTGAACGCATGACAAACTTTGATAAAAATCTTCCAAATGAAAAAGTACTACCAA
AACATAGTTTGCTTTATGAGTATTTTACGGTTTATAACGAATTGACAAAGGTCAAAT
ATGTTACTGAAGGAATGCGAAAACCAGCATTTCTTTCAGGTGAACAGAAGAAAGCC
ATTGTTGATTTACTCTTCAAAACAAATCGAAAAGTAACCGTTAAGCAATTAAAAGAA
GATTATTTCAAAAAAATAGAATGTTTTGATAGTGTTGAAATTTCAGGAGTTGAAGAT
AGATTTAATGCTTCATTAGGTACCTACCATGATTTGCTAAAAATTATTAAAGATAAA
GATTTTTTGGATAATGAAGAAAATGAAGATATCTTAGAGGATATTGTTTTAACATTG
ACCTTATTTGAAGATAGGGAGATGATTGAGGAAAGACTTAAAACATATGCTCACCTC
TTTGATGATAAGGTGATGAAACAGCTTAAACGTCGCCGTTATACTGGTTGGGGACGT
TTGTCTCGAAAATTGATTAATGGTATTAGGGATAAGCAATCTGGCAAAACAATATTA
GATTTTTTGAAATCAGATGGTTTTGCCAATCGCAATTTTATGCAGCTGATCCATGATG
ATAGTTTGACATTTAAAGAAGACATTCAAAAAGCACAAGTGTCTGGACAAGGCGAT
AGTTTACATGAACATATTGCAAATTTAGCTGGTAGCCCTGCTATTAAAAAAGGTATT
TTACAGACTGTAAAAGTTGTTGATGAATTGGTCAAAGTAATGGGGCGGCATAAGCC
AGAAAATATCGTTATTGAAATGGCACGTGAAAATCAGACAACTCAAAAGGGCCAGA
AAAATTCGCGAGAGCGTATGAAACGAATCGAAGAAGGTATCAAAGAATTAGGAAGT
CAGATTCTTAAAGAGCATCCTGTTGAAAATACTCAATTGCAAAATGAAAAGCTCTAT
CTCTATTATCTCCAAAATGGAAGAGACATGTATGTGGACCAAGAATTAGATATTAAT
CGTTTAAGTGATTATGATGTCGATCACATTGTTCCACAAAGTTTCCTTAAAGACGATT
CAATAGACAATAAGGTCTTAACGCGTTCTGATAAAAATCGTGGTAAATCGGATAAC
GTTCCAAGTGAAGAAGTAGTCAAAAAGATGAAAAACTATTGGAGACAACTTCTAAA
CGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCC
AAATCACTAAGCATGTGGCACAAATTTTGGATAGTCGCATGAATACTAAATACGATG
AAAATGATAAACTTATTCGAGAGGTTAAAGTGATTACCTTAAAATCTAAATTAGTTT
CTGACTTCCGAAAAGATTTCCAATTCTATAAAGTACGTGAGATTAACAATTACCATC
ATGCCCATGATGCGTATCTAAATGCCGTCGTTGGAACTGCTTTGATTAAGAAATATC
CAAAACTTGAATCGGAGTTTGTCTATGGTGATTATAAAGTTTATGATGTTCGTAAAA
TGATTGCTAAGTCTGAGCAAGAAATAGGCAAAGCAACCGCAAAATATTTCTTTTACT
CTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGG
CGAGATTTTGCCACAGTGCGCAAAGTATTGTCCATGCCCCAAGTCAATATTGTCAAG
AAAACAGAAGTACAGACAGGCGGATTCTCCAAGGAGTCAATTTTACCAAAAAGAAA
TTCGGACAAGCTTATTGCTCGTAAAAAAGACTGGGATCCAAAAAAATATGGTGGTTT
TGATAGTCCAACGGTAGCTTATTCAGTCCTAGTGGTTGCTAAGGTGGAAAAAGGGA
AATCGAAGAAGTTAAAATCCGTTAAAGAGTTACTAGGGATCACAATTATGGAAAGA
AGTTCCTTTGAAAAAAATCCGATTGACTTTTTAGAAGCTAAAGGATATAAGGAAGTT
AAAAAAGACTTAATCATTAAACTACCTAAATATAGTCTTTTTGAGTTAGAAAACGGT
CGTAAACGGATGCTGGCTAGTGCCGGAGAATTACAAAAAGGAAATGAGCTGGCTCT
GCCAAGCAAATATGTGAATTTTTTATATTTAGCTAGTCATTATGAAAAGTTGAAGGG
TAGTCCAGAAGATAACGAACAAAAACAATTGTTTGTGGAGCAGCATAAGCATTATT
TAGATGAGATTATTGAGCAAATCAGTGAATTTTCTAAGCGTGTTATTTTAGCAGATG
CCAATTTAGATAAAGTTCTTAGTGCATATAACAAACATAGAGACAAACCAATACGT
GAACAAGCAGAAAATATTATTCATTTATTTACGTTGACGAATCTTGGAGCTCCCGCT
GCTTTTAAATATTTTGATACAACAATTGATCGTAAACGATATACGTCTACAAAAGAA
GTTTTAGATGCCACTCTTATCCATCAATCCATCACTGGTCTTTATGAAACACGCATTG
ATTTGAGTCAGCTAGGAGGTGACTAACTCGAGTAAGGATCTCCAGGCATCAAATAA
AACGAAAGGCTCAGTCGAAAGACTGGGCCTTTCGTTTTATCTGTTGTTTGTCGGTGA
ACGCTCTCTACTAGAGTCACACTGGCTCACCTTCGGGTGGGCCTTTCTGCGTTTATAC
CTAGGGATATATTCCGCTTCCTCGCTCACTGACTCGCTACGCTCGGTCGTTCGACTGC
GGCGAGCGGAAATGGCTTACGAACGGGGCGGAGATTTCCTGGAAGATGCCAGGAAG
ATACTTAACAGGGAAGTGAGAGGGCCGCGGCAAAGCCGTTTTTCCATAGGCTCCGC
CCCCCTGACAAGCATCACGAAATCTGACGCTCAAATCAGTGGTGGCGAAACCCGAC
AGGACTATAAAGATACCAGGCGTTTCCCCCTGGCGGCTCCCTCGTGCGCTCTCCTGT
TCCTGCCTTTCGGTTTACCGGTGTCATTCCGCTGTTATGGCCGCGTTTGTCTCATTCC
ACGCCTGACACTCAGTTCCGGGTAGGCAGTTCGCTCCAAGCTGGACTGTATGCACGA
ACCCCCCGTTCAGTCCGACCGCTGCGCCTTATCCGGTAACTATCGTCTTGAGTCCAA
CCCGGAAAGACATGCAAAAGCACCACTGGCAGCAGCCACTGGTAATTGATTTAGAG
GAGTTAGTCTTGAAGTCATGCGCCGGTTAAGGCTAAACTGAAAGGACAAGTTTTGGT
GACTGCGCTCCTCCAAGCCAGTTACCTCGGTTCAAAGAGTTGGTAGCTCAGAGAACC
TTCGAAAAACCGCCCTGCAAGGCGGTTTTTTCGTTTTCAGAGCAAGAGATTACGCGC
AGACCAAAACGATCTCAAGAAGATCATCTTATTAATCAGATAAAATATTTCTAGATT
TCAGTGCAATTTATCTCTTCAAATGTAGCACCTGAAGTCAGCCCCATACGATATAAG
TTGTTACTAGTGCTTGGATTCTCACCAATAAAAAACGCCCGGCGGCAACCGAGCGTT
CTGAACAAATCCAGATGGAGTTCTGAGGTCATTACTGGATCTATCAACAGGAGTCCA
AGCGAGCTCGATATCAAATTACGCCCCGCCCTGCCACTCATCGCAGTACTGTTGTAA
TTCATTAAGCATTCTGCCGACATGGAAGCCATCACAAACGGCATGATGAACCTGAAT
CGCCAGCGGCATCAGCACCTTGTCGCCTTGCGTATAATATTTGCCCATGGTGAAAAC
GGGGGCGAAGAAGTTGTCCATATTGGCCACGTTTAAATCAAAACTGGTGAAACTCA
CCCAGGGATTGGCTGAGACGAAAAACATATTCTCAATAAACCCTTTAGGGAAATAG
GCCAGGTTTTCACCGTAACACGCCACATCTTGCGAATATATGTGTAGAAACTGCCGG
AAATCGTCGTGGTATTCACTCCAGAGCGATGAAAACGTTTCAGTTTGCTCATGGAAA
ACGGTGTAACAAGGGTGAACACTATCCCATATCACCAGCTCACCGTCTTTCATTGCC
ATACGAAATTCCGGATGAGCATTCATCAGGCGGGCAAGAATGTGAATAAAGGCCGG
ATAAAACTTGTGCTTATTTTTCTTTACGGTCTTTAAAAAGGCCGTAATATCCAGCTGA
ACGGTCTGGTTATAGGTACATTGAGCAACTGACTGAAATGCCTCAAAATGTTCTTTA
CGATGCCATTGGGATATATCAACGGTGGTATATCCAGTGATTTTTTTCTCCATTTTAG
CTTCCTTAGCTCCTGAAAATCTCGATAACTCAAAAAATACGCCCGGTAGTGATCTTA
TTTCATTATGGTGAAAGTTGGAACCTCTTACGTGCCGATCAACGTCTCATTTTCGCCA
GATATC
pSECRETS-B (SEQ ID NO: 100):
GACGTCTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATAGCT
TGTCGTAATAATGGCGGCATACTATCAGTAGTAGGTGTTTCCCTTTCTTCTTTAGCGA
CTTGATGCTCTTGATCTTCCAATACGCAACCTAAAGTAAAATGCCCCACAGCGCTGA
GTGCATATAATGCATTCTCTAGTGAAAAACCTTGTTGGCATAAAAAGGCTAATTGAT
TTTCGAGAGTTTCATACTGTTTTTCTGTAGGCCGTGTACCTAAATGTACTTTTGCTCC
ATCGCGATGACTTAGTAAAGCACATCTAAAACTTTTAGCGTTATTACGTAAAAAATC
TTGCCAGCTTTCCCCTTCTAAAGGGCAAAAGTGAGTATGGTGCCTATCTAACATCTC
AATGGCTAAGGCGTCGAGCAAAGCCCGCTTATTTTTTACATGCCAATACAATGTAGG
CTGCTCTACACCTAGCTTCTGGGCGAGTTTACGGGTTGTTAAACCTTCGATTCCGACC
TCATTAAGCAGCTCTAATGCGCTGTTAATCACTTTACTTTTATCTAAACGAGACATAC
TCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCG
AAAAGTGCCACCTGACGTCCTCTGGCTAACTAGAGAACCCACTGCTTACTGGCTTAT
CGAAATTTCCCTATCAGTGATAGAGATTGACATCCCTATCAGTGATAGAGATACTGA
GCACGTGAGACCCATGCCATAGCGTTGTTTAGGGATAACAGGGTAATACTGTCCACA
CAATCTGCCCTGGTCTCCGTTTTAGAGCTAGAAATAGCAAGTTAAAATAAGGCTAGT
CCGTTATCAACTTGAAAAAGTGGCACCGAGTCGGTGCTTTTTTGGCCGGCATGGTCC
CAGCCTCCTCGCTGGCGCCGGCTGGGCAACATGCTTCGGCATGGCGAATGGGACGA
CATCACCTCCCACAACGAAGACTACACCATCGTTGAACAGTACGAACGTGCTGAAG
GTCGTCACTCCACCGGTGCTTAAGGATCCAAACTCGAGTAAGGATCTCCAGGCATCA
AATAAAACGAAAGGCTCAGTCGAAAGACTGGGCCTTTCGTTTTATCTGTTGTTTGTC
GGTGAACGCTCTCTACTAGAGTCACACTGGCTCACCTTCGGGTGGGCCTTTCTGCGT
TTATACCTAGGGTACGGGTTTTGCTGCCCGCAAACGGGCTGTTCTGGTGTTGCTAGTT
TGTTATCAGAATCGCAGATCCGGCTTCAGCCGGTTTGCCGGCTGAAAGCGCTATTTC
TTCCAGAATTGCCATGATTTTTTCCCCACGGGAGGCGTCACTGGCTCCCGTGTTGTCG
GCAGCTTTGATTCGATAAGCAGCATCGCCTGTTTCAGGCTGTCTATGTGTGACTGTTG
AGCTGTAACAAGTTGTCTCAGGTGTTCAATTTCATGTTCTAGTTGCTTTGTTTTACTG
GTTTCACCTGTTCTATTAGGTGTTACATGCTGTTCATCTGTTACATTGTCGATCTGTTC
ATGGTGAACAGCTTTGAATGCACCAAAAACTCGTAAAAGCTCTGATGTATCTATCTT
TTTTACACCGTTTTCATCTGTGCATATGGACAGTTTTCCCTTTGATATGTAACGGTGA
ACAGTTGTTCTACTTTTGTTTGTTAGTCTTGATGCTTCACTGATAGATACAAGAGCCA
TAAGAACCTCAGATCCTTCCGTATTTAGCCAGTATGTTCTCTAGTGTGGTTCGTTGTT
TTTGCGTGAGCCATGAGAACGAACCATTGAGATCATACTTACTTTGCATGTCACTCA
AAAATTTTGCCTCAAAACTGGTGAGCTGAATTTTTGCAGTTAAAGCATCGTGTAGTG
TTTTTCTTAGTCCGTTATGTAGGTAGGAATCTGATGTAATGGTTGTTGGTATTTTGTC
ACCATTCATTTTTATCTGGTTGTTCTCAAGTTCGGTTACGAGATCCATTTGTCTATCT
AGTTCAACTTGGAAAATCAACGTATCAGTCGGGCGGCCTCGCTTATCAACCACCAAT
TTCATATTGCTGTAAGTGTTTAAATCTTTACTTATTGGTTTCAAAACCCATTGGTTAA
GCCTTTTAAACTCATGGTAGTTATTTTCAAGCATTAACATGAACTTAAATTCATCAAG
GCTAATCTCTATATTTGCCTTGTGAGTTTTCTTTTGTGTTAGTTCTTTTAATAACCACT
CATAAATCCTCATAGAGTATTTGTTTTCAAAAGACTTAACATGTTCCAGATTATATTT
TATGAATTTTTTTAACTGGAAAAGATAAGGCAATATCTCTTCACTAAAAACTAATTC
TAATTTTTCGCTTGAGAACTTGGCATAGTTTGTCCACTGGAAAATCTCAAAGCCTTTA
ACCAAAGGATTCCTGATTTCCACAGTTCTCGTCATCAGCTCTCTGGTTGCTTTAGCTA
ATACACCATAAGCATTTTCCCTACTGATGTTCATCATCTGAGCGTATTGGTTATAAGT
GAACGATACCGTCCGTTCTTTCCTTGTAGGGTTTTCAATCGTGGGGTTGAGTAGTGCC
ACACAGCATAAAATTAGCTTGGTTTCATGCTCCGTTAAGTCATAGCGACTAATCGCT
AGTTCATTTGCTTTGAAAACAACTAATTCAGACATACATCTCAATTGGTCTAGGTGA
TTTTAATCACTATACCAATTGAGATGGGCTAGTCAATGATAATTACTAGTCCTTTTCC
CGGGTGATCTGGGTATCTGTAAATTCTGCTAGACCTTTGCTGGAAAACTTGTAAATT
CTGCTAGACCCTCTGTAAATTCCGCTAGACCTTTGTGTGTTTTTTTTGTTTATATTCAA
GTGGTTATAATTTATAGAATAAAGAAAGAATAAAAAAAGATAAAAAGAATAGATCC
CAGCCCTGTGTATAACTCACTACTTTAGTCAGTTCCGCAGTATTACAAAAGGATGTC
GCAAACGCTGTTTGCTCCTCTACAAAACAGACCTTAAAACCCTAAAGGCTTAAGTAG
CACCCTCGCAAGCTCGGGCAAATCGCTGAATATTCCTTTTGTCTCCGACCATCAGGC
ACCTGAGTCGCTGTCTTTTTCGTGACATTCAGTTCGCTGCGCTCACGGCTCTGGCAGT
GAATGGGGGTAAATGGCACTACAGGCGCCTTTTATGGATTCATGCAAGGAAACTAC
CCATAATACAAGAAAAGCCCGTCACGGGCTTCTCAGGGCGTTTTATGGCGGGTCTGC
TATGTGGTGCTATCTGACTTTTTGCTGTTCAGCAGTTCCTGCCCTCTGATTTTCCAGTC
TGACCACTTCGGATTATCCCGTGACAGGTCATTCAGACTGGCTAATGCACCCAGTAA
GGCAGCGGTATCATCAACAGGCTTACCCGTCTTACTGTCCCTAGTGCTTGGATTCTC
ACCAATAAAAAACGCCCGGCGGCAACCGAGCGTTCTGAACAAATCCAGATGGAGTT
CTGAGGTCATTACTGGATCTATCAACAGGAGTCCAAGCGAGCTCTCGAACCCCAGA
GTCCCGCTCAGAAGAACTCGTCAAGAAGGCGATAGAAGGCGATGCGCTGCGAATCG
GGAGCGGCGATACCGTAAAGCACGAGGAAGCGGTCAGCCCATTCGCCGCCAAGCTC
TTCAGCAATATCACGGGTAGCCAACGCTATGTCCTGATAGCGGTCCGCCACACCCAG
CCGGCCACAGTCGATGAATCCAGAAAAGCGGCCATTTTCCACCATGATATTCGGCA
AGCAGGCATCGCCATGGGTCACGACGAGATCCTCGCCGTCGGGCATGCGCGCCTTG
AGCCTGGCGAACAGTTCGGCTGGCGCGAGCCCCTGATGCTCTTCGTCCAGATCATCC
TGATCGACAAGACCGGCTTCCATCCGAGTACGTGCTCGCTCGATGCGATGTTTCGCT
TGGTGGTCGAATGGGCAGGTAGCCGGATCAAGCGTATGCAGCCGCCGCATTGCATC
AGCCATGATGGATACTTTCTCGGCAGGAGCAAGGTGAGATGACAGGAGATCCTGCC
CCGGCACTTCGCCCAATAGCAGCCAGTCCCTTCCCGCTTCAGTGACAACGTCGAGCA
CAGCTGCGCAAGGAACGCCCGTCGTGGCCAGCCACGATAGCCGCGCTGCCTCGTCC
TGCAGTTCATTCAGGGCACCGGACAGGTCGGTCTTGACAAAAAGAACCGGGCGCCC
CTGCGCTGACAGCCGGAACACGGCGGCATCAGAGCAGCCGATTGTCTGTTGTGCCC
AGTCATAGCCGAATAGCCTCTCCACCCAAGCGGCCGGAGAACCTGCGTGCAATCCA
TCTTGTTCAATCATGCGAAACGATCCTCATCCTGTCTCTTGATCAGATCATGATCCCC
TGCGCCATCAGATCCTTGGCGGCAAGAAAGCCATCCAGTTTACTTTGCAGGGCTTCC
CAACCTTACCAGAGGGCGCCCCAGCTGGCAATTCC
pSECRETS-C (p11-LacY-wtx1, commercially available at
n2t.net/addgene:69056) (SEQ ID NO: 101):
TCGATGCATAATGTGCCTGTCAAATGGACGAAGCAGGGATTCTGCAAACCCTATGCT
ACTCCGTCAAGCCGTCAATTGTCTGATTCGTTACCAATTATGACAACTTGACGGCTA
CATCATTCACTTTTTCTTCACAACCGGCACGGAACTCGCTCGGGCTGGCCCCGGTGC
ATTTTTTAAATACCCGCGAGAAATAGAGTTGATCGTCAAAACCAACATTGCGACCGA
CGGTGGCGATAGGCATCCGGGTGGTGCTCAAAAGCAGCTTCGCCTGGCTGATACGTT
GGTCCTCGCGCCAGCTTAAGACGCTAATCCCTAACTGCTGGCGGAAAAGATGTGAC
AGACGCGACGGCGACAAGCAAACATGCTGTGCGACGCTGGCGATATCAAAATTGCT
GTCTGCCAGGTGATCGCTGATGTACTGACAAGCCTCGCGTACCCGATTATCCATCGG
TGGATGGAGCGACTCGTTAATCGCTTCCATGCGCCGCAGTAACAATTGCTCAAGCAG
ATTTATCGCCAGCAGCTCCGAATAGCGCCCTTCCCCTTGCCCGGCGTTAATGATTTG
CCCAAACAGGTCGCTGAAATGCGGCTGGTGCGCTTCATCCGGGCGAAAGAACCCCG
TATTGGCAAATATTGACGGCCAGTTAAGCCATTCATGCCAGTAGGCGCGCGGACGA
AAGTAAACCCACTGGTGATACCATTCGCGAGCCTCCGGATGACGACCGTAGTGATG
AATCTCTCCTGGCGGGAACAGCAAAATATCACCCGGTCGGCAAACAAATTCTCGTCC
CTGATTTTTCACCACCCCCTGACCGCGAATGGTGAGATTGAGAATATAACCTTTCAT
TCCCAGCGGTCGGTCGATAAAAAAATCGAGATAACCGTTGGCCTCAATCGGCGTTA
AACCCGCCACCAGATGGGCATTAAACGAGTATCCCGGCAGCAGGGGATCATTTTGC
GCTTCAGCCATACTTTTCATACTCCCGCCATTCAGAGAAGAAACCAATTGTCCATAT
TGCATCAGACATTGCCGTCACTGCGTCTTTTACTGGCTCTTCTCGCTAACCAAACCGG
TAACCCCGCTTATTAAAAGCATTCTGTAACAAAGCGGGACCAAAGCCATGACAAAA
ACGCGTAACAAAAGTGTCTATAATCACGGCAGAAAAGTCCACATTGATTATTTGCAC
GGCGTCACACTTTGCTATGCCATAGCATTTTTATCCATAAGATTAGCGGATCCTACCT
GACGCTTTTTATCGCAACTCTCTACTGTTTCTCCATACCCGTTTTTTTGGGCTAGCGA
TTGAAAACGATGCAGTTTAAGGTTTACACCTATAAAAGAGAGAGCCGTTATCGTCTG
TTTGTGGATGTACAGAGTGATATTATTGACACGCCCGGGCGACGGATGGTGATCCCC
CTGGCCAGTGCACGTCTGCTGTCAGATAAAGTCTCCCGTGAACTTTACCCGGTGGTG
CATATCGGGGATGAAAGCTGGCGCATGATGACCACCGATATGGCCAGTGTGCCGGT
CTCCGTTATCGGGGAAGAAGTGGCTGATCTCAGCCACCGCGAAAATGACATCAAAA
ACGCCATTAACCTGATGTTTTGGGGAATATAATCTAGCATTACGCTAGGGATAACAG
GGTAATATCACGCTCTAGACATACGGCATGCAAGCTTGGCTGTTTTGGCGGATGAGA
GAAGATTTTCAGCCTGATACAGATTAAATCAGAACGCAGAAGCGGTCTGATAAAAC
AGAATTTGCCTGGCGGCAGTAGCGCGGTGGTCCCACCTGACCCCATGCCGAACTCA
GAAGTGAAACGCCGTAGCGCCGATGGTAGTGTGGGGTCTCCCCATGCGAGAGTAGG
GAACTGCCAGGCATCAAATAAAACGAAAGGCTCAGTCGAAAGACTGGGCCTTTCGT
TTTATCTGTTGTTTGTCGGTGAACGCTCTCCTGAGTAGGACAAATCCGCCGGGAGCG
GATTTGAACGTTGCGAAGCAACGGCCCGGAGGGTGGCGGGCAGGACGCCCGCCATA
AACTGCCAGGCATCAAATTAAGCAGAAGGCCATCCTGACGGATGGCCTTTTTGCGTT
TCTACAAACTCTTTTGTTTATTTTTCTAAATACATTCAAATATGTATCCGCTCATGAG
ACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCT
CACCCAGAAACGCTGGTGAAAGTAAAAGATGCTGAAGATCAGTTGGGTGCACGAGT
GGGTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTATC
CCGTGTTGACGCCGGGCAAGAGCAACTCGGTCGCCGCATACACTATTCTCAGAATG
ACTTGGTTGAGTACTCACCAGTCACAGAAAAGCATCTTACGGATGGCATGACAGTA
AGAGAATTATGCAGTGCTGCCATAACCATGAGTGATAACACTGCGGCCAACTTACTT
CTGACAACGATCGGAGGACCGAAGGAGCTAACCGCTTTTTTGCACAACATGGGGGA
TCATGTAACTCGCCTTGATCGTTGGGAACCGGAGCTGAATGAAGCCATACCAAACG
ACGAGCGTGACACCACGATGCCTGCAGCAATGGCAACAACGTTGCGCAAACTATTA
ACTGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTGCT
GATAAATCTGGAGCCGGTGAGCGTGGGTCTCGCGGTATCATTGCAGCACTGGGGCC
AGATGGTAAGCCCTCCCGTATCGTAGTTATCTACACGACGGGGAGTCAGGCAACTAT
GGATGAACGAAATAGACAGATCGCTGAGATAGGTGCCTCACTGATTAAGCATTGGT
AACTGTCAGACCAAGTTTACTCATATATACTTTAGATTGATTTACGCGCCCTGTAGC
GGCGCATTAAGCGCGGCGGGTGTGGTGGTTACGCGCAGCGTGACCGCTACACTTGC
CAGCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTGCTT
TACGGCACCTCGACCCCAAAAAACTTGATTTGGGTGATGGTTCACGTAGTGGGCCAT
CGCCCTGATAGACGGTTTTTCGCCCTTTGACGTTGGAGTCCACGTTCTTTAATAGTGG
ACTCTTGTTCCAAACTTGAACAACACTCAACCCTATCTCGGGCTATTCTTTTGATTTA
TAAGGGATTTTGCCGATTTCGGCCTATTGGTTAAAAAATGAGCTGATTTAACAAAAA
TTTAACGCGAATTTTAACAAAATATTAACGTTTACAATTTAAAAGGATCTAGGTGAA
GATCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGCGC
GTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAACTCTGTA
GCACCGCCTACATACCTCGCTCTGCTAATCCTGTTACCAGTGGCTGCTGCCAGTGGC
GATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCT
ACACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGCTCA
CATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGA
GGAAGCGGAAGAGCGCCTGATGCGGTATTTTCTCCTTACGCATCTGTGCGGTATTTC
ACACCGCATACGTACGATTTAAATAGGCCTGACTCACTATAGGGAGACCGGAATTCC
CTGGCACGACAGGTTTCCCGACTGGAAAGCGGGCAGTGAGCGCAACGCAATTAATG
TGAGTTAGCTCACTCATTAGGGACCCCGGGCTTTACACTTTATGCTTCCGGCTCGTAT
GTTGTGTGGAATTGTGAGCGGATAACAATTTCACACAGGAAACAGCTATGACCATG
ATTACGGATTCACTGGCCGTCGTTTTACAACGTCGTGACTGGTAAAACCCGGGCGTT
ACCCAACTTAATCGCCTTGCAGCACATCCCCCTTTCGCCAGCAGGCGTAATAAGGAA
AGGATCCATGTACTATTTGAAAAACACAAACTTTTGGATGTTCGGTTTATTCTTTTTC
TTTTACTTTTTTATCATGGGAGCCTACTTCCCGTTTTTCCCGATTTGGCTACATGATAT
CAACCATATCAGCAAAAGTGATACGGGTATTATTTTTGCCGCTATTTCTCTGTTCTCG
CTATTATTCCAACCGCTGTTTGGTCTGCTTTCTGACAAACTCGGTCTACGCAAATACC
TGCTGTGGATTATTACCGGCATGTTAGTGATGTTTGCGCCGTTCTTTATTTTTATCTTC
GGGCCACTGCTGCAGTACAACATTTTAGTAGGATCGATTGTTGGTGGTATTTATCTA
GGCTTTAGTTTTAACGCCGGTGCGCCAGCAGTAGAGGCATTTATTGAGAAAGTCAGC
CGGCGCAGTAATTTCGAATTTGGTCGCGCGCGGATGTTTGGCAGTGTTGGCTGGGCG
CTGGTTGCCTCGATTGTCGGGATCATGTTCACCATTAATAATCAGTTTGTTTTCTGGC
TGGGCTCTGGCAGTTGTCTCATCCTCGCCGTTTTACTCTTTTTCGCCAAAACGGACGC
GCCCTCGAGTGCCACGGTTGCCAATGCGGTAGGTGCCAACCATTCGGCATTTAGCCT
TAAGCTGGCACTGGAACTGTTCAGACAGCCAAAACTGTGGTTTTTGTCACTGTATGT
TATTGGCGTTTCCTCCACCTACGATGTTTTTGACCAACAGTTTGCTAATTTCTTTACTT
CGTTCTTTGCTACCGGTGAACAGGGTACCCGCGTATTTGGCTACGTAACGACAATGG
GCGAATTACTTAACGCCTCGATTATGTTCTTTGCGCCACTGATCATTAATCGCATCGG
TGGGAAGAATGCCCTGCTGCTGGCTGGCACTATTATGTCTGTACGTATTATTGGCTC
ATCGTTCGCCACCTCAGCGCTGGAAGTGGTTATTCTGAAAACGCTGCATATGTTTGA
AGTACCGTTCCTGCTGGTGGGCTCCTTTAAATATATTACTAGTCAGTTTGAAGTGCGT
TTTTCAGCGACGATTTATCTGGTCAGTTTCAGCTTCTTTAAGCAACTGGCGATGATTT
TTATGTCTGTACTGGCGGGCAATATGTATGAAAGCATAGGTTTCCAAGGCGCTTATC
TGGTGCTGGGTCTGGTGGCGCTGGGCTTCACCTTAATTTCCGTGTTCACGCTTAGCGG
CCCGGGCCCGCTTTCCCTGCTGCGTCGTCAGGTGAATGAAGTCGCTTAAAGGCC

Cell Lines and E. coli Strains

All cloning was performed using New England Biolabs (NEB) 10-beta cells (NEB #C3020K) or TOP10 (Invitrogen #C404010) cells, and all SECRETS assays performed in Stbl2 cells (Invitrogen #10268019), grown at 30 °C. A549 (ATCC CCL-185™) human lung epithelial cells were obtained from were obtained from ATCC (American Type Culture Collection).

Cloning SECRETS Plasmids and x-gRNA Libraries:

### SECRETS Plasmids:

Three plasmids were generated for the validation of the SECRETS protocol: pSECRETS-A (medium copy p15A ori, chloramphenicol resistance, aTc-inducible Cas9 expression; FIG. 9A ii), pSECRETS-B (low copy SC101 ori, kanamycin resistance, aTc-inducible gRNA expression, and a site for “off-target” sequence; FIG. 9A iii), and pSECRETS-C (p11.LacY.wtx1 plasmid (Addgene #69056) high copy pBR322 ori, ampicillin resistance, arabinose-inducible/glucose-suppressed ccdB toxin also containing additional site for “target” sequence; FIG. 9A i). p11-LacY-wtx1 was a gift from Huimin Zhao (Addgene plasmid #69056; n2t.net/addgene: 69056; RRID: Addgene_69056).

To clone pSECRETS-A, the Cas9 gene was PCR amplified from pwtCas9-bacteria (Addgene #44250) and a gBlock (purchased from Integrated DNA Technologies [IDT]) containing an anhydrotetracycline (aTc) inducible promoter (pLTetO-1) were inserted via HiFi Assembly (HiFi Assembly Kit (NEB #E5520S)) into a PCR-ed fragment of plasmid pBbA2c-RFP (Addgene #35326) to replace the red fluorescent protein (RFP) gene. pwtCas9-bacteria was a gift from Stanley Qi (Addgene plasmid #44250; n2t.net/addgene: 44250; RRID: Addgene_44250). pBbA2c-RFP was a gift from Jay Keasling (Addgene plasmid #35326; n2t.net/addgene: 35326; RRID: Addgene_35326).

To clone pSECRETS-B, the gRNA cassette for aTc-induced expression was constructed by inserting via HiFi Assembly a PCR'ed fragment of TetR from pBbA2c-RFP (Addgene #35326) and a gBlock (IDT) containing the pLTetO-1 promoter and a Golden Gate cassette (dual BsaI restriction sites) near a Cas9 fused tracrRNA-crRNA fusion to replace the RFP and LacI genes in a PCR'ed fragment of pBbS2K-RFP (Addgene #35330). The Golden Gate assembly cassettes of the resulting plasmid (called pSECRETS-B) could then be used to clone spacer sequences or x-gRNA libraries for gRNA expression after BsaI digestion and ligation of short phosphorylated annealed oligos or HiFi Assembly of single-stranded oligos, respectively. pBbS2k-RFP was a gift from Jay Keasling (Addgene plasmid #35330; n2t.net/addgene: 35330; RRID: Addgene_35330)

To clone pSECRETS-B derivatives containing off-target sequences and standard gRNAs or x-gRNA libraries, single-stranded oligonucleotides (oPools) were synthesized by IDT containing the off-target sequences, pLTetO-1 promoter, (N8 random nucleotides immediately upstream) the spacer sequence. These oligos were of the form of SEQ ID NO: 104:
5′-CCACTGCTTACTGGCTTATCGGAAGGGATCGTCCTGACCCCG [Off-target sequence, 20 nt+3 nt PAM] CCCCCTCCGTGGAGAAAATTTCCCTATCAGTGATAGAGATTGACATCCCTATCAGTG ATAGAGATACTGAGCAC [5′-extension library, for example: NNNNNNNN] [20 nt spacer sequence] GTTTTAGAGCTAGAAATAGCAAG-3′.

These inserts were PCR'ed with primers:
SECRETS-FwdUSER 5′-AGCAAG\deoxyU\TAAAATAAGGCTAGTCCG-3′ and
SECRETS-RevUSER 5′-ACTTGC\deoxyU\ATTTCTAGCTCTAAAAC-3′
   where deoxyU is a deoxyuracil modified, and the plasmid pSECRETS-B PCR'ed with primers:
Bv3-FwdUSER 5′-AGCAAG\deoxyU\TAAAATAAGGCTAGTCCG-3′ and
B-RevUSER 5′-AGTGGG\deoxyU\TCTCTAGTTAGCCAGAG-3′

These inserts were then cloned into the pSECRETS-B cassette via USER cloning (NEB #M5505S). To maintain library diversity, after transformation, E. coli was recovered in 1 mL SOC media for 1 hour without selection, then 0.5 mL of the media was reinoculated into 7 mL LB with kanamycin (50 μg/mL) and grown overnight. 5 mL of the transformants were then centrifuged, then miniprepped. pSECRETS-B plasmids were sequenced using variants of primers:
SECRETS-BSeq5 5′-[NGS adapter] [barcode]-GAGCGGATACATATTTGAATG-3′
SECRETS-BSeq3 5′-[NGS adapter] [barcode]-AAGTTGATAACGGACTAGCC-3′

The pSECRETS-C plasmids containing desired on-target sequences were constructed via HiFi assembly into the p11.LacY.wtx1 plasmid (Addgene #69056), which was double digested with XbaI and SphI, with a dsDNA fragment containing a target site (20 bp+PAM), 15 bp genomic context sequences flanking the target, and overhang sequences matching the digested plasmid. These fragments were constructed by PCRing short oligos with the form:
5′-ATAACAGGGTAATATCACGC
[15 bp upstream genomic sequence context]+
[20 bp target sequence+3 bp PAM]+
[15 bp downstream genomic sequence context]
AAGCTTGGCTGTTTTGGCGG-3′

E. coli strains containing pSECRETS-C plasmids were grown in solutions supplemented with glucose (glu) to suppress leakage of arabinose-induced promoter until selection.

### The SECRETS Protocol and Analysis

Validating Selection Strength Using Standard gRNAs

For validation, pSECRETS-C itself or pSECRETS-C containing the EMX1 target site and flanking sequences (pSECRETS-C-EMX1); pSECRETS-A; and pSECRETS-B to express the standard EMX1 gRNA (pSECRETS-B-EMX1-gRNA) or pSECRETS-B-EMX1-gRNA containing with EMX1 OT1 were electroporated sequentially into electrocompetent NEB10beta E. coli cells and recovered in SOC media. For the last transformation with pSECRETS-B, recovery media was supplemented with 10 ng/ml aTc for pre-induction of sgRNA and Cas9. Following recoveragcy for 1 hr, cells were plated on LB agar plates under selective (aTc, arabinose, chloramphenicol, kanamycin) and non-selective (glucose, chloramphenicol, kanamycin, ampicillin) conditions and incubated for 24 hours.

Selection of Extended g-RNAs (SECRETS Protocol)

pSECRETS-B plasmids containing x-gRNA libraries and the off-target site were screened similarly to the validation experiments with few changes. E. coli cells were transformed in two steps instead of three: E. coli strains containing pSECRETS-A plasmids were electroporated with corresponding B and C plasmid simultaneously (75 ng each DNA). Following recovery, cells were centrifuged at 4 °C and supernatant was replaced with fresh LB before inoculating 0.5 mL of the culture into 7 mL liquid LB for selective or non-selective conditions and grown overnight. After miniprep (NEB #T1010L) of the resulting cultures, samples were PCR'ed across the gRNA segment and prepared for Illumina next-generation sequencing using variants of primers SECRETS-BSeq5 and SECRETS-BSeq3.

### Analysis of SECRETS Outcomes

Small-scale (at least 50,000 reads) next-generation (Amplicon-EZ; Azenta Inc.) was performed of samples from the SECRETS assay. Custom code was written in MATLAB (Mathworks; Natick, MA) to extract and count the 5′-extensions from the x-gRNA sequence of each read; however, in principle, a short line of code can be written to the same effect following the approach found in: Maxwell, C. S., Jacobsen, T., Marshall, R., Noireaux, V. & Beisel, C. L. A detailed cell-free transcription-translation-based assay to decipher CRISPR protospacer-adjacent motifs. Methods 143, 48-57 (2018). In short, the extension sequences were found by identifying sequences with perfect identity to 20 bp of the gRNA promoter and the 20 bp spacer sequences, and if the spacing between the two sequences matched the expected length of the extension sequence, those sequences were recorded. The number of unique 5′-extensions were enumerated per sample and normalized to the total number of reads per sample and averaged across technical replicates (n=2). The normalized number of reads per 5′-extension were then averaged across biological replicates (n=2), sorted from most prevalent to least, and the top five most prevalent 5′-extensions per gRNA selected for further characterization.

In Vitro Validation of x-gRNAs

### Cas9 Ribonucleoprotein (RNP) Generation:

DNA oligos of sgRNAs and x-gRNAs were designed according to the EnGen sgRNA Synthesis Kit (NEB #E3322) to add 5′-T7 RNA polymerase promoter sequence and 3′-Cas9 crRNA sequence and were purchased from Integrated DNA Technologies IDT then resuspended to a stock concentration of 100 μM. If the (x-)gRNA did not have an initial 5′-dG necessary for T7 RNA polymerase transcription, one was added in the DNA oligo sequence. For sgRNA synthesis, oligos were diluted 100× (1 μM) then used with the EnGen sgRNA Synthesis Kit per manufacturer's instructions. Cas9 RNPs were formed following the IDT Alt-R CRISPR-Cas9 System—In vitro cleavage of target DNA with ribonucleoprotein complex protocol (Option 2). Cas9 enzyme (Sigma Aldrich, #CAS9PROT-250UG), eCas9 enzyme (Sigma Aldrich #ESPCAS9PRO-50UG), or dCas9 enzyme (IDT Alt-R® S.p. dCas9 Protein V3 #1081066) and sgRNA were combined in equimolar amounts in Phosphate buffered saline, pH 7.4-PBS (ThermoFisher, #10010023) and incubated at room temperature for 10 minutes. Following incubation, RNPs were stored at −80 °C or immediately used for in vitro digestion reactions.

### In Vitro Digestion Reactions:

Three hundred (300) bp DNA targets containing the target sequence ~200 bp from one end and the flanking genomic context were synthesized by Twist Bioscience, PCR amplified using the provided universal primers, purified, and resuspended in nuclease-free water to 100 nM. Three technical replications of reactions were assembled in the following order: 7 μL nuclease-free water, 1 μL target DNA substrate (100 nM), 1 μL 10× Cas9 Nuclease Reaction buffer (200 mM HEPES, 1 M NaCl, 50 mM MgCl2, 1 mM EDTA (pH 6.5 at 25 °C)), 1 μL Cas9-RNP (1 mM), then incubated for 1 hour at 37 °C followed by proteinase K digestion (1 μL−56 °C for 10 minutes; ThermoFisher, #EO0491). Products were resolved on a 3% agarose gel stained with SYBR Gold and analyzed using ImageJ.

Evaluation of Gene Activity of Transfected Cas9/x-gRNAs into Human Cell Lines

Cells were transfected using the Lipofectamine CRISPRMAX Cas9 Transfection Reagent (ThermoFisher #CMAX00003) kit. Prior to transfection, A549 fibroblast cells were plated in 24-well plates at 25% confluency in Dulbecco's Modified Eagle's Medium-DMEM (ATCC #30-2002)+10% Fetal Bovine Serum-FBS (ATCC 30-2020)+1% Penicillin-Streptomycin solution (ATCC 30-2300) and incubated for 24 hours at 37 °C+5% CO2. Following incubation, the media was removed and cells were washed with 1×PBS and replaced with fresh DMEM+10% FBS. Cas9 RNP complexes were formed in a 1:1.2 molar ratio of Cas9 protein to sgRNA with Cas9 Plus reagent to a total volume of 25 μL in Opti-MEM Reduced Serum Medium (ThermoFisher #31985070) per reaction (n=3). RNPs were added to a mix of 25 μL Opti-MEM I and 1.5 μL CRISPRMAX reagent per reaction, and following a 10 minute room temperature incubation, 50 μL was added to each well. Cells were then incubated at 37 °C+5% CO2 for 48 hours.

### Analysis of Gene Editing Outcomes:

Cells were processed as follows using the GeneArt Genomic Cleavage Detection Kit (ThermoFisher #A24372). Cell media was collected in a 1.5 mL Eppendorf tube. Remaining attached cells were washed with PBS then detached using TrypLe Express (ATCC 30-2300) trypsin and transferred to the corresponding Eppendorf tube for centrifugation at 1,200×g at 4 °C to pellet cells. Once the supernatant was discarded, pellets were resuspended in cell lysis buffer with protein degrader (supplied in kit) and incubated at 68 °C then 95 °C to lyse cells. Crude cell lysate was mixed with forward and reverse primer (10 μM), AmpliTaq Gold 360 master mix, and nuclease-free water for direct PCR amplification of the region of interest followed by agarose gel electrophoresis to confirm expected PCR length. Heteroduplexes of the PCR products were formed by mixing with 10× detection buffer and heating samples to 95 °C, cooling to 85 °C at 2 °C/sec, then to 25 °C at 0.1 °C/sec. Detection enzyme was added to the samples and incubated at 37 °C, then fragments were resolved on a 3% agarose gel stained with SYBR Gold. Fluorescence was measured through ImageJ, intensity normalized by length of the DNA fragments, and fraction cleaved was determined using the following equation:

[sum of cleaved band intensities/(sum of cleaved and parental band intensities)]×100%

Two technical replicates of samples were also prepared for illumina next-generation sequencing and amplicon sequencing with editing efficiency determined using the CRISPResso2 pipeline.

### Genome-Wide Off-Target Screens:

Genome-wide off-target editing was measured using CHANGE-seq (See Lazzarotto, C. R. et al. CHANGE-seq reveals genetic and epigenetic effects on CRISPR-Cas9 genome-wide activity. Nature Biotechnology, 1-11 (2020)). The genomic DNA (human male/female mixed, Promega #G3041) purification steps were carried out using the NEB Monarch Genomic DNA Purification Kit. An agarose gel was used to visualize the tagmentation of the human genomic DNA with the transposase. For the PCR step after cleavage and USER enzyme treatment (step 25 of the supplemental information), NEB 2X Q5 Master Mix was used in place of 2× Kapa HiFi HotStart Ready Mix. In place of the MiSeq protocol described in the supplemental information, cleaved genomic DNA barcoded and amplified via PCR for illumina sequencing was sent for NGS Amplicon-EZ sequencing by Azenta, and analyzed using the CHANGE-seq computational pipeline (github.com/tsailabSJ/changeseq) with default settings (i.e., >4 reads minimum/hit).

### Availability of Materials:

pSECRETS-A and pSECRETS-B precursors (without gRNA spacers or off-targets) will be provided to Addgene plasmid repository. pSECRETS-C precursor plasmids (without on-target sequences) are available from Addgene (Addgene plasmid #69056; n2t.net/addgene: 69056; RRID: Addgene_69056) as a gift from Huimin Zhao.

### Results

To overcome this challenge, here is presented an experimental protocol to screen tens- to hundreds- of thousands of candidate 5′-extension sequences simultaneously to efficiently and reliably identify novel to extended gRNA sequences (x-gRNAs) that maintain robust Cas9 activity on-target while significantly increasing their gene editing specificity by effectively eliminating their activity even at known off-target sequences where conventional approaches to increase Cas9 specificity in general may fail (FIG. 9). In this protocol, called “Selection of Extended CRISPR RNAs with Enhanced Targeting and Specificity” (SECRETS), the activities of Cas9 enzymes with a library of x-gRNA candidates are evaluated in parallel using an Escherichia coli strain that is strongly selective for their ability to stimulate Cas9 nuclease activity on-target and strongly counter-selective for activity at their off-targets. The SECRETS E. coli strain maintains three plasmids (FIG. 9A): (i) a high-copy plasmid expresses the toxin ccdB in the presence of arabinose (ara), and also contains the target sequence of interest; (ii) a medium-copy plasmid expresses Cas9 in the presence of anhydrotetracycline (aTc); and (iii) a low-copy plasmid expresses the gRNA of interest, provides resistance to the antibiotic kanamycin (KanR), and also contains a known off-target sequence for the gRNA. DSBs induced by the Cas9 at the target of interest in E. coli results in the degradation of ccdB plasmid, allowing the bacteria to survive in the presence of arabinose, while DSBs induced by the Cas9 at the known off-target results in the degradation of the gRNA plasmid and bacterial susceptibility to the antibiotic kanamycin (KanS). Hence, only gRNAs that exhibit robust activity at their intended targets (degrading the high-copy toxin plasmid) and low activity at their off-target sites (not degrading the low-copy gRNA plasmid) will survive a SECRETS screen (FIG. 9C).

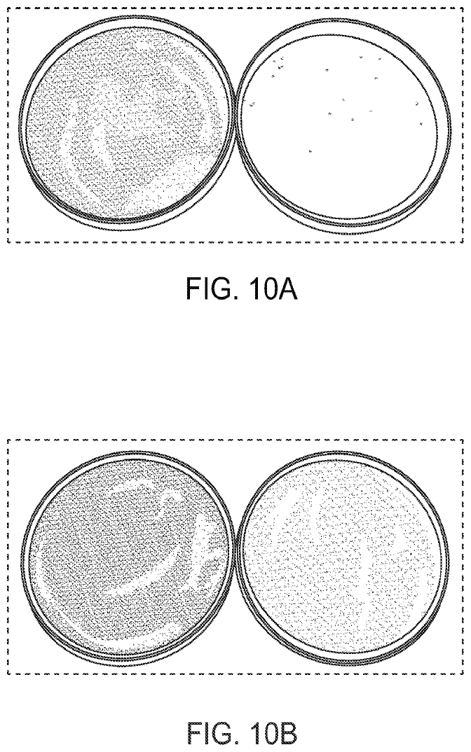

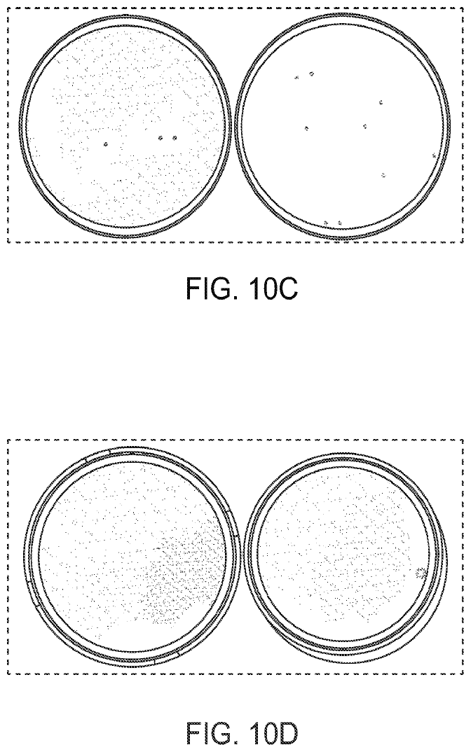

This system was tested with a well-characterized gRNA for human gene EMX1, its target sequence (EMX1 ON), and another sequence found in the human genome where the Cas9/gRNA RNP complex is known to exhibit off-target activity (EMX1 OFF1) due to a two nucleotide difference with the EMX1 ON target at positions where Cas9 effectors are especially susceptible to tolerating sequence divergence (FIG. 9A). After only 1 hr of Cas9/gRNA expression, followed by plating and overnight growth on LB with aTc, arabinose, chloramphenicol (cam), and kanamycin, strong suppression of E. coli growth was found with the standard EMX1 gRNA, but only when the EMX1 OFF1 sequence was present in the kanamycin resistance plasmid (FIG. 10).

As shown in FIG. 10A, the SECRETS screen is demonstrated with pSECRETS-A, an empty toxin plasmid (pSECRETS-C with no target), and pSECRETS-B with an EMX1 sgRNA after plating on non-selective (left) vs selective (right) conditions. As shown in FIG. 10B, the SECRETS screen is demonstrated with pSECRETS-A, pSECRETS-C with EMX1 ON, and pSECRETS-B with an EMX1 sgRNA on non-selective (left) vs selective (right) conditions. As shown in FIG. 10C, the SECRETS screen is demonstrated with pSECRETS-A, pSECRETS-C with EMX1 ON, and pSECRETS-B with an EMX1 sgRNA and EMX1 OFF1 on non-selective (left) vs selective (right) conditions. And finally, as shown in FIG. 10D, the SECRETS screen is demonstrated with pSECRETS-A, pSECRETS-C with EMX1 ON, and pSECRETS-B with an EMX1 x-gRNA library (N8) and EMX1 OFF1 on non-selective (left) vs selective (right) conditions.

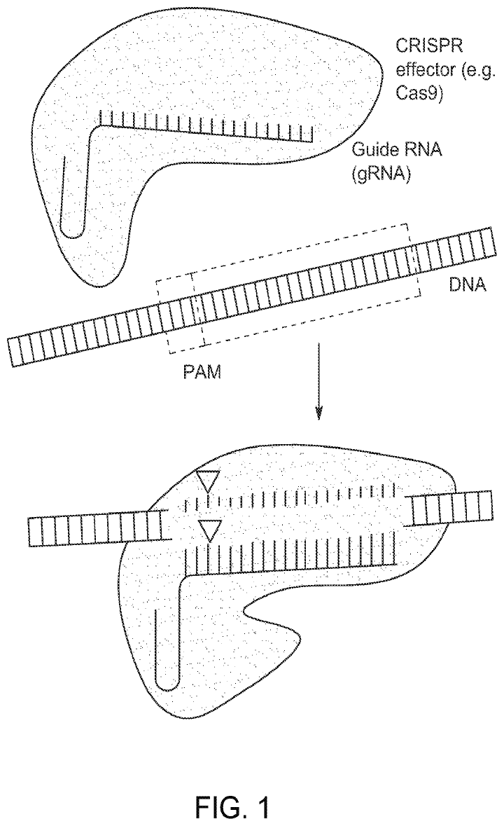

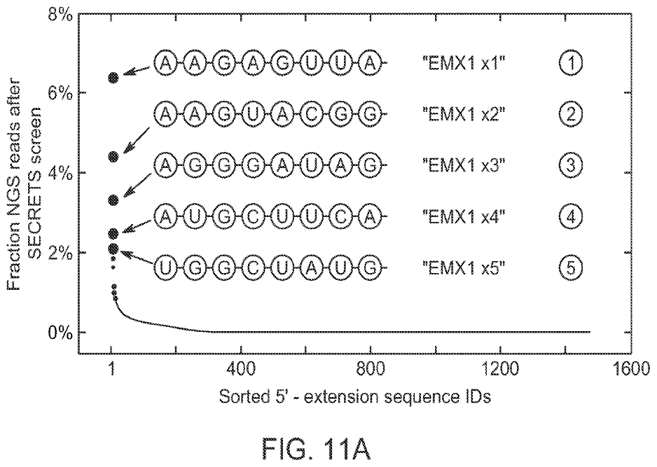

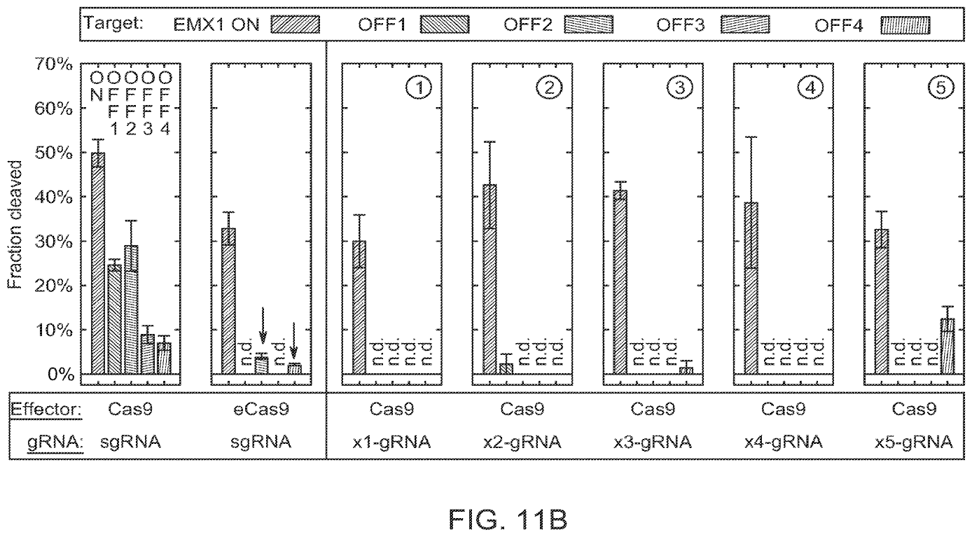

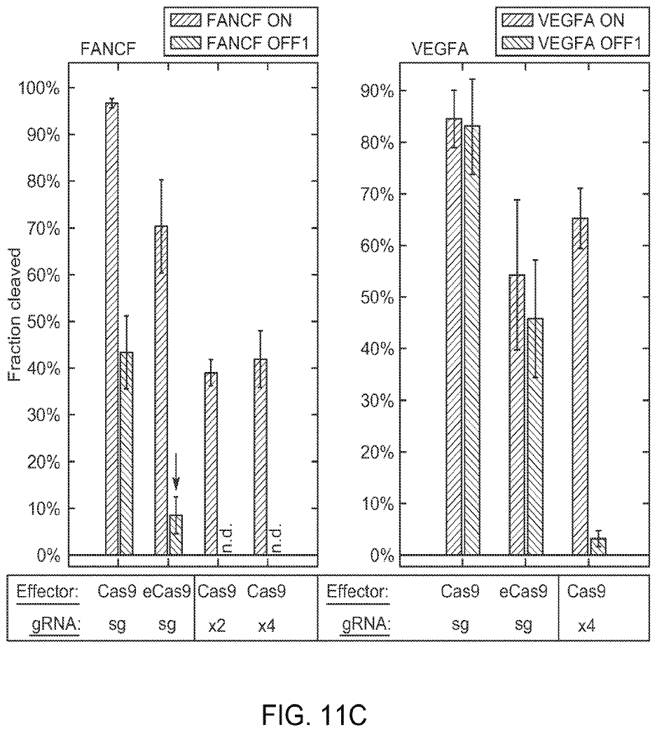

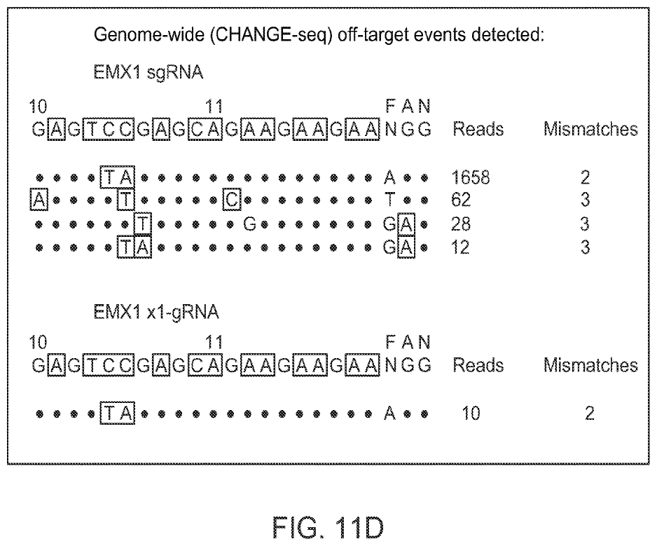

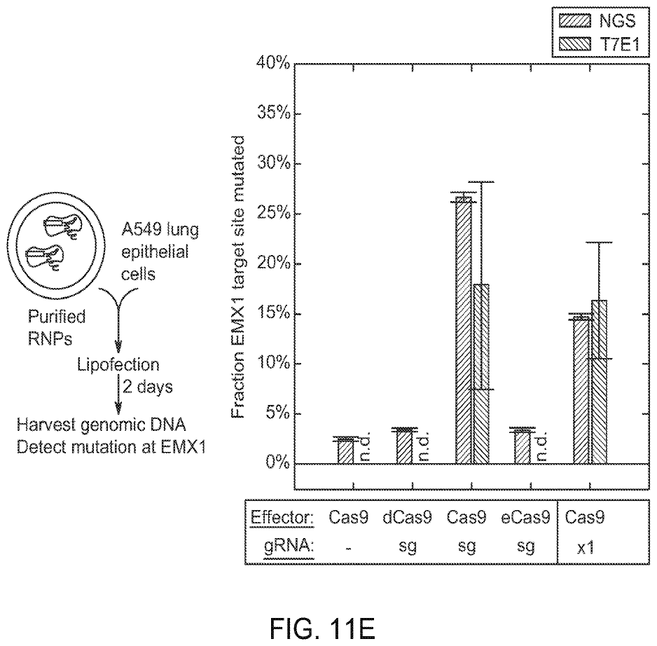

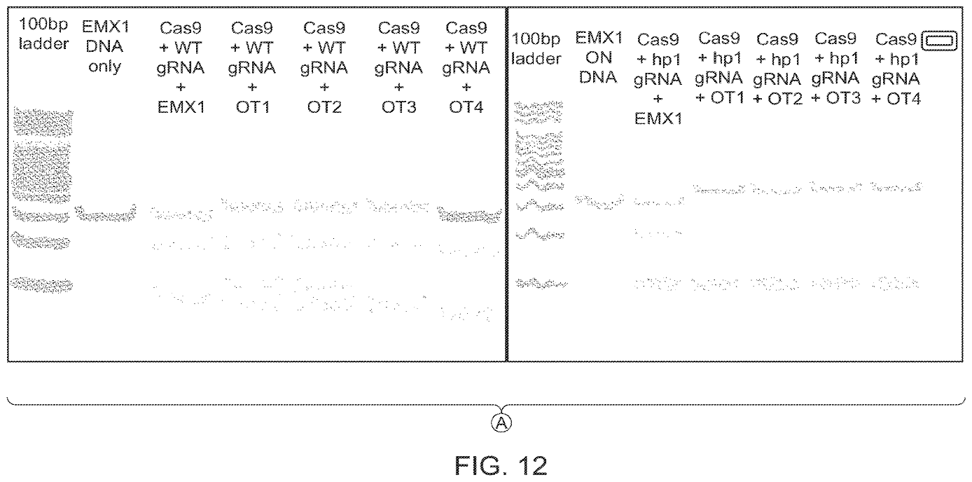

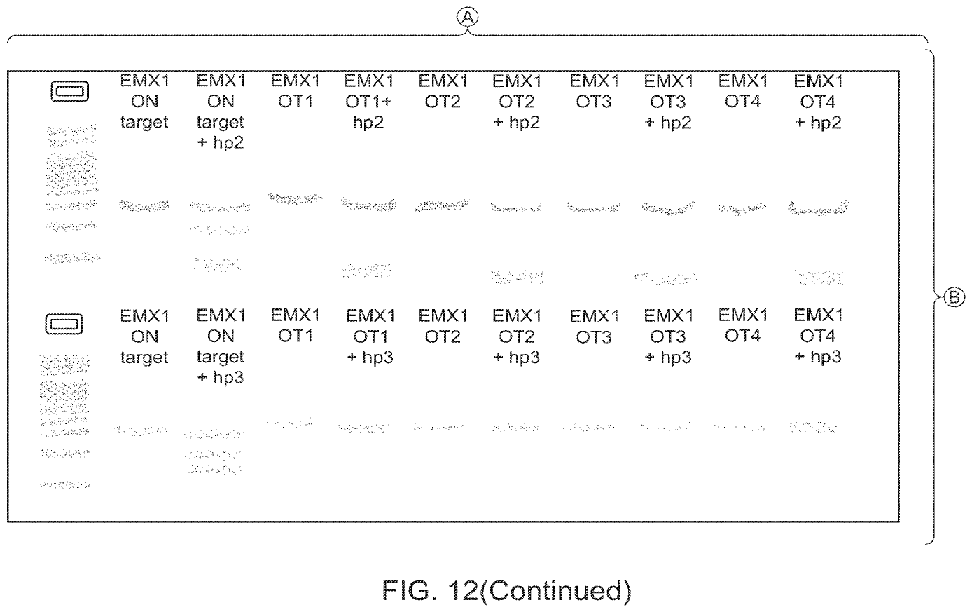

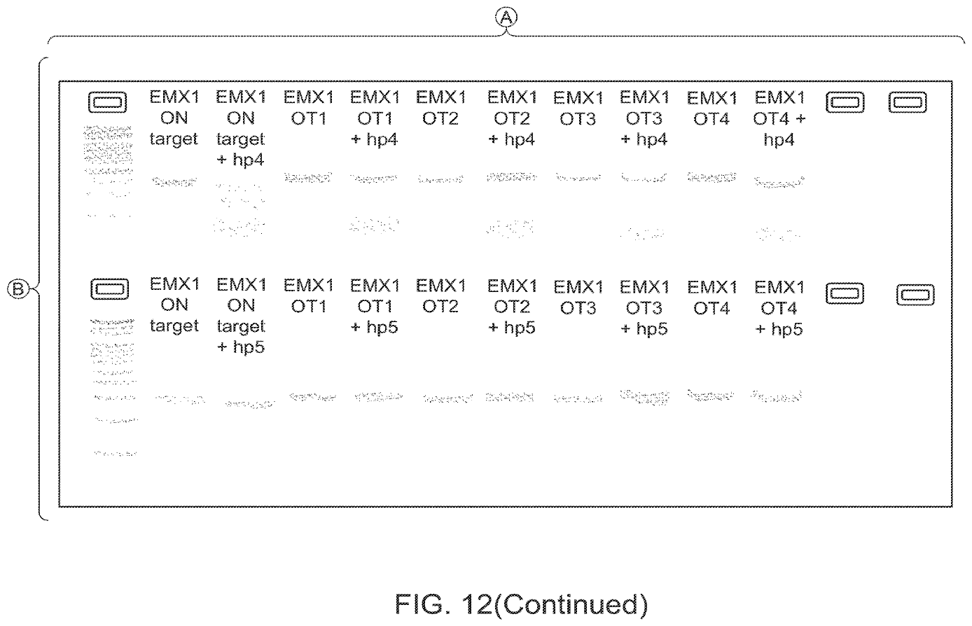

If instead of the standard gRNA for EMX1 (FIG. 9B top) a library of EMX1 x-gRNA variants with 8 randomized nucleotides (N8) appended to its 5′ end (FIG. 1B bottom) was introduced, numerous E. coli colonies of survivors of the SECRETS protocol are found (FIG. 9D and FIG. 10), indicating that these x-gRNAs from the library demonstrate the high Cas9 activity and specificity required to survive. The pooled survivors were sequenced and each of the top five most prevalent x-gRNA sequences in the surviving population (FIG. 11A) were tested for activity and specificity in vitro (FIG. 11B and FIG. 12, Table 5).

TABLE 5
Extensions from SECRETS protocol for EMX1, FANCF, and VEGFA targets and top
off-target. (top five extensions sequences labeled as in FIG. 11).

| EMX1 | | VEGFA | | FANCF | |
|---|---|---|---|---|---|
| 5′- extension | Fraction | 5′- extension | Fraction | 5′- extension | Fraction |
| sequences | reads | sequences | reads | sequences | reads |
| ATGCTTCA E [symbol] | 0.063642 | GGCCCAGC | 0.022094 | ACATTCCT F [symbol] | 0.028027 |
| | | V [symbol] | | | |
| AAGTACGG | 0.044337 | ATCGCGAT | 0.015392 | AAAGATAG | 0.01289 |
| E [symbol] | | V [symbol] | | F [symbol] | |
| AAGAGTTA | 0.033594 | TTTGGAAT | 0.013769 | AATTACTT F [symbol] | 0.007923 |
| E [symbol] | | V [symbol] | | | |
| AGGGATAG | 0.024857 | TCTCACCA | 0.011858 | CCAAGTCT F [symbol] | 0.007864 |
| E [symbol] | | V [symbol] | | | |
| TGGCTATG | 0.020792 | GGGCCCTC | 0.0089 | ATTGGTGA | 0.0068 |
| E [symbol] | | V [symbol] | | F [symbol] | |
| CAATAAAG | 0.018731 | GGTGTGGC | 0.008508 | GTCGTTAC | 0.005144 |
| AAAGTCTG | 0.018671 | CCTTTGAC | 0.008036 | ACGACGTA | 0.004849 |
| GAGATCAG | 0.016166 | CTACTCCA | 0.007487 | GAAGATTG | 0.004789 |
| ATGGCAGG | 0.011847 | TAACTATC | 0.007356 | CAATAAAA | 0.004435 |
| GTTAGTAG | 0.011538 | CCGCCGAA | 0.00733 | GAGTGCTC | 0.004316 |
| ACGAGTGT | 0.011184 | TGCGTAGC | 0.006937 | GGGTGAAC | 0.004316 |
| TGCGTGAG | 0.009982 | TGAACTAA | 0.006701 | ATGGCCGG | 0.004198 |
| CGTGGTAT | 0.009542 | TATGTCCC | 0.006335 | GCCCACAA | 0.00408 |
| GATTAGCG | 0.0095 | TCGACTTC | 0.006021 | ATAGGTGT | 0.003548 |
| AGTGGATC | 0.009345 | CCGCATCC | 0.005942 | GGCCATAC | 0.003548 |
| AAACTAGC | 0.008774 | TGGGGCGC | 0.005942 | GTGCAATT | 0.003548 |
| GGTGAGAG | 0.008427 | CCTCCTGC | 0.005864 | GACGAGCC | 0.00343 |
| TCTCGGTG | 0.007407 | TTCATTTA | 0.005811 | GAGGCGTT | 0.003311 |
| TAAATTTT | 0.00716 | CCCTTTCA | 0.005759 | GAGGTAAC | 0.003311 |
| TGGTCCTC | 0.007007 | TGCCTGCA | 0.005733 | GTCACGCT | 0.003311 |
| GGTATTTT | 0.006975 | ATGCATAG | 0.005654 | ATTCGAGT | 0.003252 |
| GGTGCATA | 0.006914 | CCAGCGAA | 0.005602 | GGCGTCTT | 0.003193 |
| GAGAGCAA | 0.006854 | GGTGAGGC | 0.005602 | GGGCACAG | 0.003193 |
| TGACCCAC | 0.006641 | TGCCCACT | 0.005366 | GGATGAGG | 0.003016 |
| GGCGCCGT | 0.006529 | CCCTTGTA | 0.005026 | CCTCTGGT | 0.002956 |
| CTGCTCCT | 0.006518 | TAATCTCT | 0.005 | GCAACCCT | 0.002956 |
| AATCAAGC | 0.006449 | CCGGCATT | 0.004895 | ATTGGTCT | 0.002838 |
| AATCACCG | 0.0064 | GATGTAGG | 0.004843 | AATAAAAC | 0.00272 |
| TTATCAGT | 0.006298 | GTACTATC | 0.004712 | ACCTTTAA | 0.00272 |
| AGATTTTC | 0.006248 | ATGCGATA | 0.00466 | GCCTGGAC | 0.002602 |
| TTTGTAGA | 0.006229 | TTCTACAC | 0.004633 | TAGTCATC | 0.002602 |
| GTATGGTG | 0.006108 | CCAGCCAT | 0.004581 | AGATGCAT | 0.002543 |
| GGCAGGTG | 0.006082 | GCTTCGCC | 0.004424 | AGGCGAGA | 0.002543 |
| GGTAAGTT | 0.006025 | CCCTGCCG | 0.004372 | ATGTTCGA | 0.002543 |
| GTGCACGT | 0.005758 | ACCCTGGC | 0.004293 | CAGATTTC | 0.002483 |
| ACAATTGA | 0.005685 | GGGCAATG | 0.004293 | CCCTATGT | 0.002483 |
| AGGTGTGA | 0.005567 | TGAGGATG | 0.004267 | GCGTAGTG | 0.002483 |
| GTCGTGCC | 0.005317 | CAGGCTCA | 0.004241 | GGTATTTT | 0.002365 |
| ATTAGAGT | 0.005264 | GGGTAGGA | 0.004215 | AGATTTAA | 0.002306 |
| CCGCCTTT | 0.005216 | CCCCCGTC | 0.004057 | GATCCGTT | 0.002247 |
| TGTGTTTC | 0.005119 | TCAGAATG | 0.003979 | GGCTAATC | 0.002247 |
| CACTGGTC | 0.004938 | CCAGCAAC | 0.003953 | GTGCTTTG | 0.002247 |
| TGACCAAG | 0.004926 | ACTAAAAA | 0.003927 | ATAGTTTT | 0.002188 |
| TAACGATG | 0.00489 | AAGCGGGA | 0.003796 | ATCATTGA | 0.002129 |
| TGACGGGT | 0.00477 | ACTAGCTC | 0.003796 | GCGCTACG | 0.002129 |
| GAGGTAGG | 0.004761 | GCGTGTGA | 0.003796 | ACTGGTGT | 0.001951 |
| GGGTTTAC | 0.004753 | GGGGTGGC | 0.00377 | ATATTGGG | 0.001951 |
| GACGTTAG | 0.004693 | CCCACTGT | 0.003691 | GACTCTAA | 0.001951 |
| TCTTTACG | 0.004604 | GTCCCCAC | 0.003691 | ATCATTTC | 0.001892 |

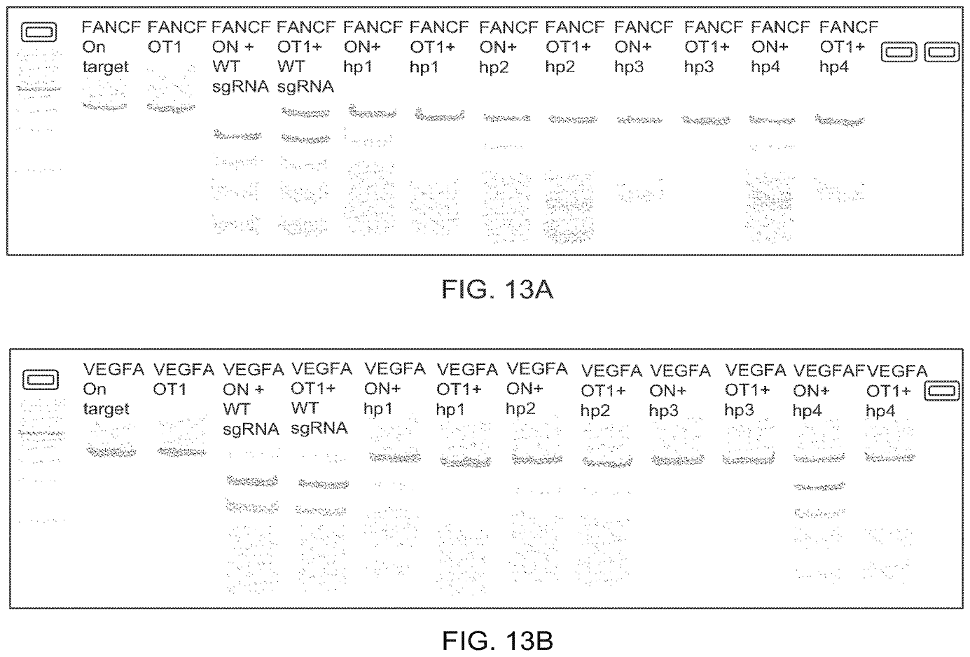

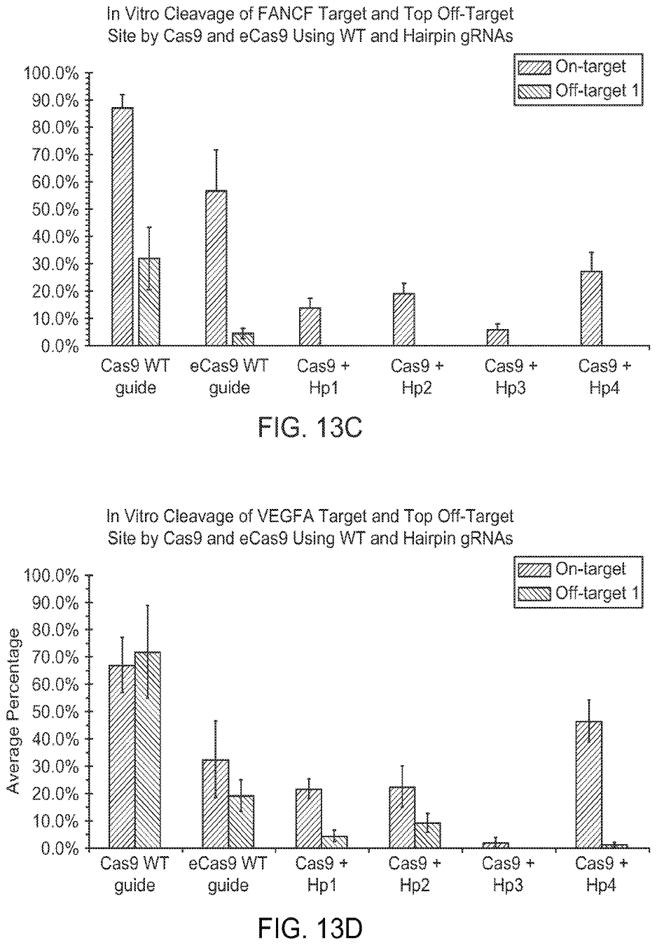

In vitro Cas9 digestion assays revealed nuclease activities of Cas9 with all five of the x-gRNAs identified from the SECRETS screen had significantly reduced off-target activity at EXM1 OFF1 compared to the standard EMX1 gRNA—effectively eliminating nuclease activity at the known off-target site—and exhibiting similar activity at EMX1 ON as the engineered “enhanced specificity” Cas9 variant eCas9. These five x-gRNAs identified through the SECRETS protocol could also exhibit higher levels of specificity in general, eliminating off-target activity across three other known EMX1 off-targets (EMX1 OFF2-OFF4, containing 2 to 4 differences with the EMX1 ON sequence) for Cas9, and reducing nuclease activity at all four off-target sequences even more so relative to eCas9 with a standard gRNA (FIG. 11B and FIG. 12). In addition to x-gRNAs for EMX1, we were also able to readily identify multiple x-gRNAs using the SECRETS protocol for other targets—human genes VEGFA and FANCF (FIG. 12, FIG. 13A-D, and Table 5)—with superior activity and specificity profiles (FIG. 11C), including in cases (VEGFA) where eCas9 was not able to significantly reduce activity at OFF1 sites.

To confirm that the x-gRNAs identified via SECRETS would remain active in human cell lines for gene editing, RNP complexes with Cas9 variants (wild-type Cas9; catalytically-inactive dCas9; or engineered enhanced-specificity eCas9) and gRNA variants (standard gRNAs or x-gRNAs) were transfected into A549 human lung epithelial cells, then T7E1 mutation detection assays and next-generation sequencing (NGS) were performed to quantify mutation rates at the EMX1 target. The Cas9 RNPs with x-gRNAs identified via SECRETS exhibited robust gene editing cells in A549 cells, and higher on-target mutation rates than eCas9 (FIG. 11E). It is noted that, even though they have additional 5′-nucleotides, hp-gRNAs had previously been found not to recognize or mutate any novel off-target sites compared to the standard gRNAs, likely because the 20 nt targeting segment of the hp-gRNA remains the same. To ensure that no new off-targets were introduced when using x-gRNAs identified from the SECRETS screen a test of genome-wide off-target nuclease activity (CHANGE-seq5) was also performed and, as expected, findings were significant reductions of off-target cleavage activity genome-wide and no new off-targets (FIG. 11D). Therefore, these findings demonstrate that the SECRETS protocol can robustly identify multiple high-performance x-gRNA candidates with strong potential for specific gene editing applications in human cells that eliminate off-target activity at selected loci.

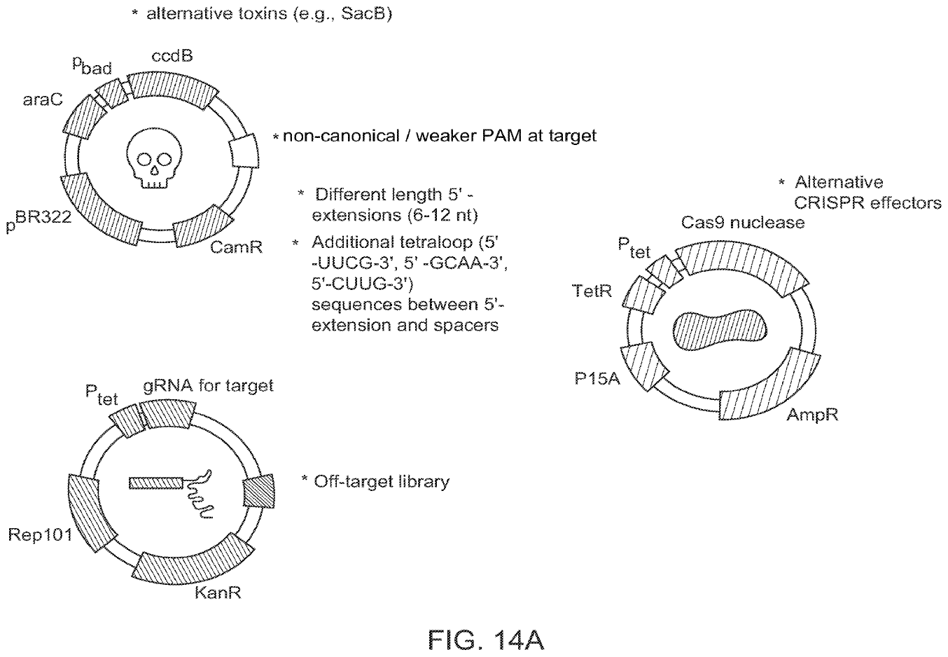

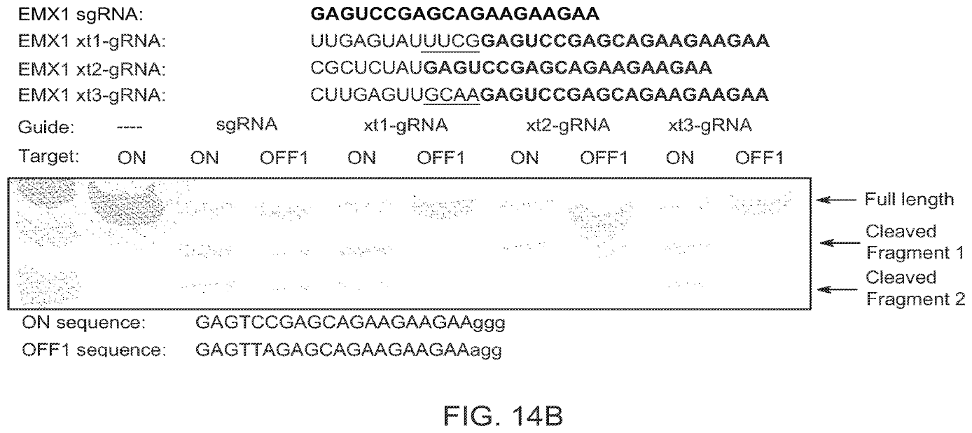

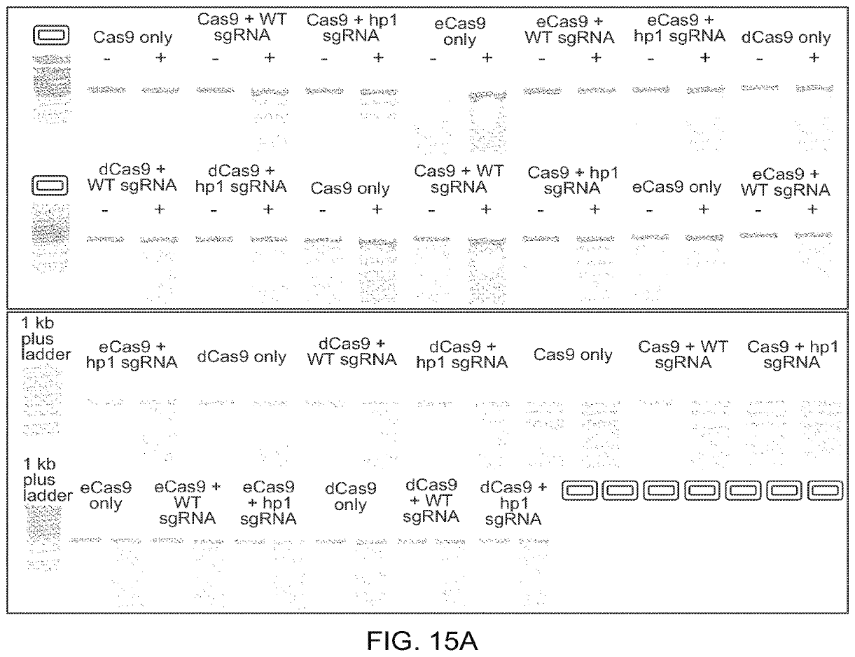

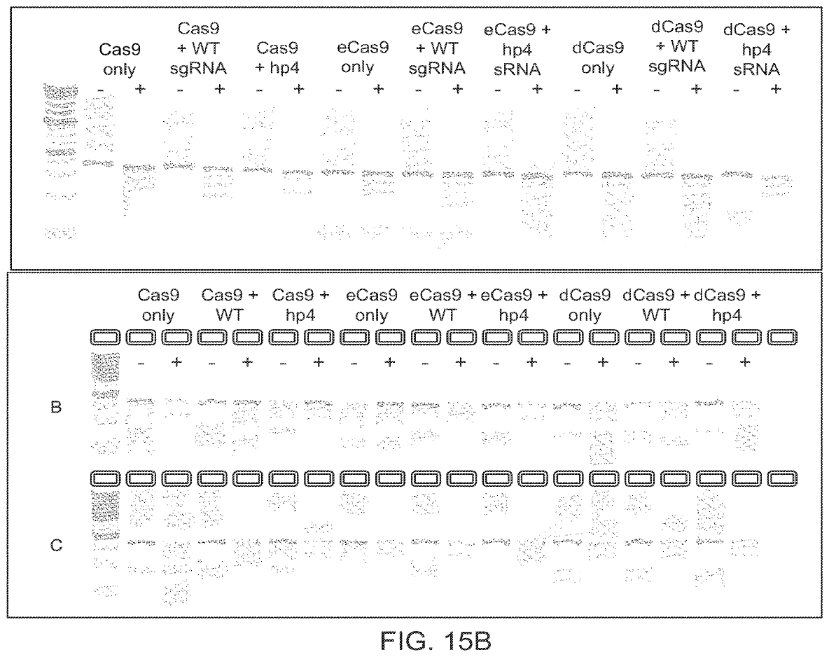

In the demonstrations above, randomized x-gRNA libraries of 65,336 variants (N8) were screened in the SECRETS protocol for each gRNA target. Enhanced x-gRNAs from more complex initial libraries (>250,000 5′-extension sequence variants) were also identified, including pooled libraries of N8 variants containing different additional 4 nt tetraloop motifs17 (N8+4) designed to promote interactions between the N8 segment and the DNA-targeting segment of the x-gRNA (FIG. 14A). Indeed, it was noted that the ‘space’ of potential 5′-extension sequences for x-gRNAs is quite large (FIG. 14)—larger x-gRNA libraries of >4\hat{ }10 or 4\hat{ }12 (>1-16M) variants are expected to be quite readily generated and screened in E. coli for new targets/off-target pairs of interest. The results here suggest that this space is also quite rich with high performance x-gRNA variants, as multiple x-gRNAs were identified for several spacer sequences under relatively small-scale SECRETS screens that exhibited exceptional activity and specificity profiles (FIG. 14B, FIG. 15A-D). Furthermore, hp-gRNAs were able to improve the specificity of diverse CRISPR effectors, including Cas9 from Staphylococcus aureus and various Cas12 effectors, the SECRETS protocol can be readily adapted to those systems as well.

For many biotechnological or therapeutic applications, it is often desirable direct a Cas9/gRNA RNP to a specific nucleotide target of interest (e.g., where there might be no flexibility to target nearby sites). If it is determined there is a potential for off-target activity at certain sites in a specific sample or for a patient, there can be limited options to limit that activity at those specific off-targets. Here it is demonstrated that the SECRETS protocol can be used to robustly identify ultra-specific variants for those gRNAs of interest that have been explicitly counter-selected against activity at those off-target sites. This approach could be used to robustly generate x-gRNAs in a “design-free” way that effectively eliminates the need for individualized optimization, and has been experimentally streamlined for simplicity in cloning new target/off-target pairs on-demand into the screening plasmids and for ease of rapidly selecting enhanced x-gRNAs. Once any off-target activity has been suspected or characterized, the SECRETS screen therefore provides an accessible and reliable method to identify high-performance gRNA variants for specific targets of interest. It is expected that the continued output and development of this approach will allow for safer applications of advanced CRISPR gene editing approaches that require gRNAs with extreme specificity, such as SNP-targeting and/or allele-specific gene editing. As approaches to predicting and identifying novel off-target sequences at the level of individual patients become more sophisticated and routine, it is expected that methods like SECRETS, which can reliably and rapidly generate highly-specific and highly-active gRNA variants that effectively eliminate Cas9 activity at specific off-targets, will become increasing important in applications of gene therapies in personalized medicine.

### Example 4

Extended gRNA for Treatment of Sickle Cell Disease

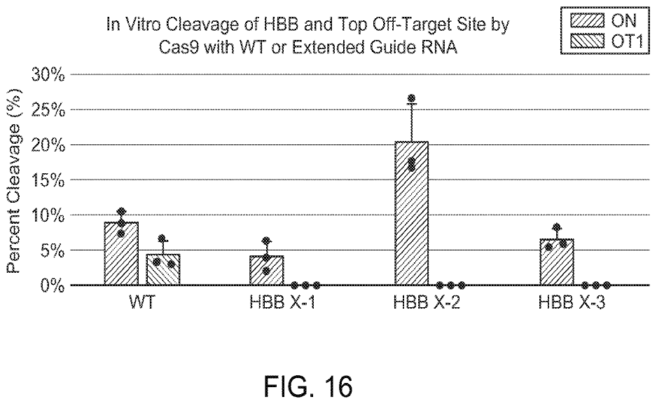

The HBB gene encodes for the hemoglobin subunit beta protein, which along with alpha globin, is the most common form of hemoglobin in adult humans. Thousands of naturally occurring variants of HBB exist, including a point mutation producing HbS, which causes sickle cell disease. Others have developed a gRNA gene therapy treatment (classic.clinicaltrials.gov/ct2/show/NCT04774536). Alternative extended gRNA were developed using the methods of the present invention denoted by HBB X-1, HBB X-2, and HBB X-3. These gRNA were compared to WT sgRNA in FIG. 16 with:
On-target sequence, ON (SEQ ID NO: 102): CTTGCCCCACAGGGCAGTAACGG
Off-target sequence, OT1 (SEQ ID NO: 103): TCAGCCCCACAGGGCAGTAAGGG

OT1 is the top off-target sequence. The extended gRNA show a better safety profile for gene therapy treatment of sickle cell disease than WT, with HBB X-2 displaying twice the on-target cleavage compared to WT, along with no off-target cleavage.

What is claimed:

1. A process for selecting crRNA sequences for CRISPR gene editing comprising:
identifying a target oligonucleotide sequence;
transferring the target oligonucleotide sequence to a first plasmid, wherein the first plasmid further comprises an oligonucleotide sequence encoding a toxin;
obtaining a second plasmid, wherein the second plasmid comprises an oligonucleotide sequence encoding a CRISPR effector;
identifying a plurality of off-target oligonucleotide sequences;
obtaining a plurality of third plasmids;
transferring each off-target oligonucleotide sequences into a different third plasmid from the plurality of third plasmids, wherein each third plasmid from the plurality of third plasmids further comprises an oligonucleotide sequence encoding a crRNA;
transfecting an organism with the first plasmid, the second plasmid, and the plurality of third plasmids, wherein,
the crRNA sequence comprises an x-gRNA structure, x portion including n-m nucleotides covalently linked to a gRNA portion, and the gRNA portion comprises a sequence of nucleotides complementary to a portion of the target oligonucleotide sequence.

2. The process of claim 1, wherein the target oligonucleotide sequence is selected from a mutation or variant sequence that results in phenotypic abnormality.

3. The process of claim 1, wherein the target oligonucleotide sequence includes a disease causing or promoting sequence.

4. The process of claim 3, wherein the disease is cancer.

5. The process of claim 3, wherein the disease is sickle cell disease.

6. The process of claim 2, wherein at least one of the plurality of off-target oligonucleotide sequences are healthy alleles.

7. The process of claim 1, wherein the CRISPR effector is Cas9.

8. The process of claim 1, wherein the organism is Escherichia coli.

9. The process of claim 1, wherein the x portion is configured to form a secondary structure with the gRNA portion.

10. The process of claim 9, wherein the secondary structure is a hairpin.

11. The process of claim 1, wherein the x portion is determined using a recombinant library.

12. A system for selecting crRNA sequences for CRISPR gene editing comprising:
a first plasmid, wherein the first plasmid comprises a target oligonucleotide sequence and a first oligonucleotide sequence encoding a toxin;
a second plasmid, wherein the second plasmid comprises a second oligonucleotide sequence encoding a CRISPR effector;
a plurality of third plasmids, wherein each third plasmid of the plurality of third plasmids comprises an off-target sequence and a third oligonucleotide sequence encoding a crRNA, wherein,
the crRNA sequence comprises an x-gRNA structure, x portion including n-m nucleotides covalently linked to a gRNA portion, and the gRNA portion comprises a sequence of nucleotides complementary to a portion of the target oligonucleotide sequence.

13. The system of claim 12, wherein the target oligonucleotide sequence is selected from a mutation or variant sequence that results in phenotypic abnormality.

14. The system of claim 12, wherein the CRISPR effector is Cas9.

15. The system of claim 12, wherein the x portion is configured to form a secondary structure with the gRNA portion.

16. The system of claim 15, wherein the secondary structure is a hairpin.

17. The system of claim 12, wherein the x portion includes 8-12 nucleotides.

18. A process for selecting crRNA sequences for CRISPR gene editing comprising:
identifying a target oligonucleotide sequence;
transferring the target oligonucleotide sequence to a first plasmid, wherein the first plasmid further comprises an oligonucleotide sequence encoding a toxin;
obtaining a second plasmid, wherein the second plasmid comprises an oligonucleotide sequence encoding a CRISPR effector;
identifying a plurality of off-target oligonucleotide sequences;
obtaining a plurality of third plasmids;
transferring each off-target oligonucleotide sequences into a different third plasmid from the plurality of third plasmids, wherein each third plasmid from the plurality of third plasmids further comprises an oligonucleotide sequence encoding a crRNA and an antibiotic resistance gene;
transfecting more than one organism with the first plasmid, the second plasmid, and the plurality of third plasmids, wherein,
the crRNA sequence comprises an x-gRNA structure, x portion including 8-12 nucleotides covalently linked to a gRNA portion, and the gRNA portion comprises a sequence of nucleotides complementary to a portion of the target oligonucleotide sequence.

19. The process of claim 18, further comprising inducing expression of the toxin and the CRISPR effector, such that only those organisms where the CRISPR effector induces double strand breaks at the target oligonucleotide sequence survive toxin induction.

20. The process of claim 18, further comprising inducing expression of the CRISPR effector and exposing the organisms to an antibiotic to which the antibiotic resistance gene confers resistance, such that only those organisms where the CRISPR effector fails to induce double strand breaks at the off-target sequences survive the antibiotic exposure.


SEQUENCE LISTING (108 sequences)

SEQ ID NO: 1 - DNA, 20 bp, synthetic construct
  source 1..20: mol_type = other DNA
agtcacactggctcaccttc

SEQ ID NO: 2 - DNA, 20 bp, synthetic construct
  source 1..20: mol_type = other DNA
taacaacccgtaaactcgcc

SEQ ID NO: 3 - DNA, 20 bp, synthetic construct
  source 1..20: mol_type = other DNA
gcacaaatagcgtcggatgg

SEQ ID NO: 4 - DNA, 20 bp, synthetic construct
  source 1..20: mol_type = other DNA
gaaggtgagccagtgtgact

SEQ ID NO: 5 - DNA, 20 bp, synthetic construct
  source 1..20: mol_type = other DNA
tcgttgtgggaggtgatgtc

SEQ ID NO: 6 - DNA, 16 bp, synthetic construct
  source 1..16: mol_type = other DNA
gacgaagactcaattg

SEQ ID NO: 7 - DNA, 20 bp, synthetic construct
  source 1..20: mol_type = other DNA
gacatcacctcccacaacga

SEQ ID NO: 8 - DNA, 20 bp, synthetic construct
  source 1..20: mol_type = other DNA
ggtaaggttgggaagccctg

SEQ ID NO: 9 - DNA, 19 bp, synthetic construct
  source 1..19: mol_type = other DNA
cctgcgccatcagatcctt

SEQ ID NO: 10 - DNA, 20 bp, synthetic construct
  source 1..20: mol_type = other DNA
ggtggagtgacgaccttcag

SEQ ID NO: 11 - DNA, 20 bp, synthetic construct
  source 1..20: mol_type = other DNA
gacatcacctcccacaacga

SEQ ID NO: 12 - DNA, 20 bp, synthetic construct
  source 1..20: mol_type = other DNA
taacaacccgtaaactcgcc

SEQ ID NO: 13 - DNA, 19 bp, synthetic construct
  source 1..19: mol_type = other DNA
aagcttggctgttttggcg

SEQ ID NO: 14 - DNA, 23 bp, synthetic construct
  source 1..23: mol_type = other DNA
gcgtgatattaccctgttatccc

SEQ ID NO: 15 - DNA, 20 bp, synthetic construct
  source 1..20: mol_type = other DNA
ataacagggtaatatcacgc

SEQ ID NO: 16 - DNA, 40 bp, synthetic construct
  source 1..40: mol_type = other DNA
ccgccaaaacagccaagcttttgatgtgatgggagctctt

SEQ ID NO: 17 - DNA, 39 bp, synthetic construct
  source 1..39: mol_type = other DNA
ccgccaaaacagccaagcttgctcggaaaagcgatctag

SEQ ID NO: 18 - DNA, 39 bp, synthetic construct
  source 1..39: mol_type = other DNA
ccgccaaaacagccaagctttcagaaatagggggtccag

SEQ ID NO: 19 - DNA, 25 bp, synthetic construct
  source 1..25: mol_type = other DNA
  modified_base 7: mod_base = OTHER; note = Deoxyuracil
agcaagntaaaataaggctagtccg

SEQ ID NO: 20 - DNA, 24 bp, synthetic construct
  source 1..24: mol_type = other DNA
  modified_base 7: mod_base = OTHER; note = Deoxyuracil
agtgggntctctagttagccagag

SEQ ID NO: 21 - DNA, 23 bp, synthetic construct
  source 1..23: mol_type = other DNA
  modified_base 7: mod_base = OTHER; note = Deoxyuracil
acccacngcttactggcttatcg

SEQ ID NO: 22 - DNA, 24 bp, synthetic construct
  source 1..24: mol_type = other DNA
  modified_base 7: mod_base = OTHER; note = Deoxyuracil
acttgcnatttctagctctaaaac

SEQ ID NO: 23 - DNA, 195 bp, synthetic construct
  source 1..195: mol_type = other DNA
ccactgcttactggcttatcggaagggcaagacagattgtcagagttagagcagaagaagaaaggcatggagtaaaggcaaaatttccctatcagtgatagagattgacatccctatcagtgatagagatactgagcacnnnnnnnngagtccgagcagaagaagaagttttagagctagaaatagcaagttaaa

SEQ ID NO: 24 - DNA, 195 bp, synthetic construct
  source 1..195: mol_type = other DNA
ccactgcttactggcttatcggaagggcaagacagattgtcagagttagagcagaagaagaatttcatggagtaaaggcaaaatttccctatcagtgatagagattgacatccctatcagtgatagagatactgagcacnnnnnnnngagtccgagcagaagaagaagttttagagctagaaatagcaagttaaa

SEQ ID NO: 25 - DNA, 190 bp, synthetic construct
  source 1..190: mol_type = other DNA
ccactgcttactggcttatcggaagggatcgtcctgaccccgggaaccccgtctgcagcaccaggccccctccgtggagaaaatttccctatcagtgatagagattgacatccctatcagtgatagagatactgagcacnnnnnnnnggaatcccttctgcagcaccgttttagagctagaaatagcaag

SEQ ID NO: 26 - DNA, 190 bp, synthetic construct
  source 1..190: mol_type = other DNA
ccactgcttactggcttatcggaagggcttcgcgcacctcatggaatcccttctgcagcacctttatcgcttttccgagcaaatttccctatcagtgatagagattgacatccctatcagtgatagagatactgagcacnnnnnnnnggaatcccttctgcagcaccgttttagagctagaaatagcaag

SEQ ID NO: 27 - DNA, 190 bp, synthetic construct
  source 1..190: mol_type = other DNA
ccactgcttactggcttatcggaagggaaggacggatttgtgggatggagggagtttgctcctggggtgtcagaatgtccaaatttccctatcagtgatagagattgacatccctatcagtgatagagatactgagcacnnnnnnnngggtggggggagtttgctccgttttagagctagaaatagcaag

SEQ ID NO: 28 - DNA, 190 bp, synthetic construct
  source 1..190: mol_type = other DNA
ccactgcttactggcttatcggaagggtgggctttggaaagggggtggggggagtttgctcctttaccccctatttctgaaaatttccctatcagtgatagagattgacatccctatcagtgatagagatactgagcacnnnnnnnngggtggggggagtttgctccgttttagagctagaaatagcaag

SEQ ID NO: 29 - DNA, 58 bp, synthetic construct
  source 1..58: mol_type = other DNA
acactctttccctacacgacgctcttccgatctttttgagcggatacatatttgaatg

SEQ ID NO: 30 - DNA, 59 bp, synthetic construct
  source 1..59: mol_type = other DNA
acactctttccctacacgacgctcttccgatctaaaaagagcggatacatatttgaatg

SEQ ID NO: 31 - DNA, 60 bp, synthetic construct
  source 1..60: mol_type = other DNA
acactctttccctacacgacgctcttccgatctggggtagagcggatacatatttgaatg

SEQ ID NO: 32 - DNA, 61 bp, synthetic construct
  source 1..61: mol_type = other DNA
acactctttccctacacgacgctcttccgatctccccgccgagcggatacatatttgaatg

SEQ ID NO: 33 - DNA, 56 bp, synthetic construct
  source 1..56: mol_type = other DNA
gactggagttcagacgtgtgctcttccgatctttttaagttgataacggactagcc

SEQ ID NO: 34 - DNA, 57 bp, synthetic construct
  source 1..57: mol_type = other DNA
gactggagttcagacgtgtgctcttccgatctaaaaaaagttgataacggactagcc

SEQ ID NO: 35 - DNA, 58 bp, synthetic construct
  source 1..58: mol_type = other DNA
gactggagttcagacgtgtgctcttccgatctggggtaaagttgataacggactagcc

SEQ ID NO: 36 - DNA, 59 bp, synthetic construct
  source 1..59: mol_type = other DNA
gactggagttcagacgtgtgctcttccgatctccccgccaagttgataacggactagcc

SEQ ID NO: 37 - DNA, 24 bp, synthetic construct
  source 1..24: mol_type = other DNA
tatgtagcctcagtcttcccatca

SEQ ID NO: 38 - DNA, 22 bp, synthetic construct
  source 1..22: mol_type = other DNA
tggttgcccaccctagtcattg

SEQ ID NO: 39 - DNA, 25 bp, synthetic construct
  source 1..25: mol_type = other DNA
gaaaactcaaagaaatgcccaatca

SEQ ID NO: 40 - DNA, 26 bp, synthetic construct
  source 1..26: mol_type = other DNA
tctgtggaggcaataaagtgaaataa

SEQ ID NO: 41 - DNA, 20 bp, synthetic construct
  source 1..20: mol_type = other DNA
gggcctggaagttcgctaat

SEQ ID NO: 42 - DNA, 20 bp, synthetic construct
  source 1..20: mol_type = other DNA
aggcgtatcatttcgcggat

SEQ ID NO: 43 - DNA, 20 bp, synthetic construct
  source 1..20: mol_type = other DNA
gttgcgtttgaggatgagca

SEQ ID NO: 44 - DNA, 21 bp, synthetic construct
  source 1..21: mol_type = other DNA
gaaatctcccacgagtctccg

SEQ ID NO: 45 - DNA, 20 bp, synthetic construct
  source 1..20: mol_type = other DNA
tccagatggcacattgtcag

SEQ ID NO: 46 - DNA, 20 bp, synthetic construct
  source 1..20: mol_type = other DNA
agggagcaggaaagtgaggt

SEQ ID NO: 47 - DNA, 20 bp, synthetic construct
  source 1..20: mol_type = other DNA
accccacagccaggttttca

SEQ ID NO: 48 - DNA, 21 bp, synthetic construct
  source 1..21: mol_type = other DNA
gaatcactgcacctggccatc

SEQ ID NO: 49 - DNA, 59 bp, synthetic construct
  source 1..59: mol_type = other DNA
acactctttccctacacgacgctcttccgatctatcacgagtttctcatctgtgcccct

SEQ ID NO: 50 - DNA, 60 bp, synthetic construct
  source 1..60: mol_type = other DNA
acactctttccctacacgacgctcttccgatctcgatgtaagtttctcatctgtgcccct

SEQ ID NO: 51 - DNA, 61 bp, synthetic construct
  source 1..61: mol_type = other DNA
acactctttccctacacgacgctcttccgatctttaggctaagtttctcatctgtgcccct

SEQ ID NO: 52 - DNA, 62 bp, synthetic construct
  source 1..62: mol_type = other DNA
acactctttccctacacgacgctcttccgatcttgaccagtaagtttctcatctgtgcccct

SEQ ID NO: 53 - DNA, 63 bp, synthetic construct
  source 1..63: mol_type = other DNA
acactctttccctacacgacgctcttccgatctacatgtcgtaagtttctcatctgtgcccct

SEQ ID NO: 54 - DNA, 64 bp, synthetic construct
  source 1..64: mol_type = other DNA
acactctttccctacacgacgctcttccgatctgccaatacgtaagtttctcatctgtgcccct

SEQ ID NO: 55 - DNA, 58 bp, synthetic construct
  source 1..58: mol_type = other DNA
gactggagttcagacgtgtgctcttccgatctcagatcatcgatgtcctccccattgg

SEQ ID NO: 56 - DNA, 58 bp, synthetic construct
  source 1..58: mol_type = other DNA
gactggagttcagacgtgtgctcttccgatctacttgaatcgatgtcctccccattgg

SEQ ID NO: 57 - DNA, 58 bp, synthetic construct
  source 1..58: mol_type = other DNA
gactggagttcagacgtgtgctcttccgatctgatcagatcgatgtcctccccattgg

SEQ ID NO: 58 - DNA, 58 bp, synthetic construct
  source 1..58: mol_type = other DNA
gactggagttcagacgtgtgctcttccgatcttagcttatcgatgtcctccccattgg

SEQ ID NO: 59 - DNA, 58 bp, synthetic construct
  source 1..58: mol_type = other DNA
gactggagttcagacgtgtgctcttccgatctggctagatcgatgtcctccccattgg

SEQ ID NO: 60 - DNA, 58 bp, synthetic construct
  source 1..58: mol_type = other DNA
gactggagttcagacgtgtgctcttccgatctcttgtaatcgatgtcctccccattgg

SEQ ID NO: 61 - DNA, 59 bp, synthetic construct
  source 1..59: mol_type = other DNA
acactctttccctacacgacgctcttccgatctatcacgcgcttgtccatgtctaggaa

SEQ ID NO: 62 - DNA, 60 bp, synthetic construct
  source 1..60: mol_type = other DNA
acactctttccctacacgacgctcttccgatctcgatgtacgcttgtccatgtctaggaa

SEQ ID NO: 63 - DNA, 61 bp, synthetic construct
  source 1..61: mol_type = other DNA
acactctttccctacacgacgctcttccgatctttaggctacgcttgtccatgtctaggaa

SEQ ID NO: 64 - DNA, 62 bp, synthetic construct
  source 1..62: mol_type = other DNA
acactctttccctacacgacgctcttccgatcttgaccagtacgcttgtccatgtctaggaa

SEQ ID NO: 65 - DNA, 63 bp, synthetic construct
  source 1..63: mol_type = other DNA
acactctttccctacacgacgctcttccgatctacatgtcgtacgcttgtccatgtctaggaa

SEQ ID NO: 66 - DNA, 64 bp, synthetic construct
  source 1..64: mol_type = other DNA
acactctttccctacacgacgctcttccgatctgccaatacgtacgcttgtccatgtctaggaa

SEQ ID NO: 67 - DNA, 58 bp, synthetic construct
  source 1..58: mol_type = other DNA
gactggagttcagacgtgtgctcttccgatctcagatctggcatggcaagacagattg

SEQ ID NO: 68 - DNA, 58 bp, synthetic construct
  source 1..58: mol_type = other DNA
gactggagttcagacgtgtgctcttccgatctacttgatggcatggcaagacagattg

SEQ ID NO: 69 - DNA, 58 bp, synthetic construct
  source 1..58: mol_type = other DNA
gactggagttcagacgtgtgctcttccgatctgatcagtggcatggcaagacagattg

SEQ ID NO: 70 - DNA, 58 bp, synthetic construct
  source 1..58: mol_type = other DNA
gactggagttcagacgtgtgctcttccgatcttagctttggcatggcaagacagattg

SEQ ID NO: 71 - DNA, 58 bp, synthetic construct
  source 1..58: mol_type = other DNA
gactggagttcagacgtgtgctcttccgatctggctagtggcatggcaagacagattg

SEQ ID NO: 72 - DNA, 58 bp, synthetic construct
  source 1..58: mol_type = other DNA
gactggagttcagacgtgtgctcttccgatctcttgtatggcatggcaagacagattg

SEQ ID NO: 73 - DNA, 58 bp, synthetic construct
  source 1..58: mol_type = other DNA
acactctttccctacacgacgctcttccgatctatcacgctccagagccgtgcgaatg

SEQ ID NO: 74 - DNA, 59 bp, synthetic construct
  source 1..59: mol_type = other DNA
acactctttccctacacgacgctcttccgatctcgatgtactccagagccgtgcgaatg

SEQ ID NO: 75 - DNA, 60 bp, synthetic construct
  source 1..60: mol_type = other DNA
acactctttccctacacgacgctcttccgatctttaggctactccagagccgtgcgaatg

SEQ ID NO: 76 - DNA, 61 bp, synthetic construct
  source 1..61: mol_type = other DNA
acactctttccctacacgacgctcttccgatcttgaccagtactccagagccgtgcgaatg

SEQ ID NO: 77 - DNA, 62 bp, synthetic construct
  source 1..62: mol_type = other DNA
acactctttccctacacgacgctcttccgatctacatgtcgtactccagagccgtgcgaatg

SEQ ID NO: 78 - DNA, 63 bp, synthetic construct
  source 1..63: mol_type = other DNA
acactctttccctacacgacgctcttccgatctgccaatacgtactccagagccgtgcgaatg

SEQ ID NO: 79 - DNA, 59 bp, synthetic construct
  source 1..59: mol_type = other DNA
gactggagttcagacgtgtgctcttccgatctcagatcaatcagtacgcagagagtcgc

SEQ ID NO: 80 - DNA, 59 bp, synthetic construct
  source 1..59: mol_type = other DNA
gactggagttcagacgtgtgctcttccgatctacttgaaatcagtacgcagagagtcgc

SEQ ID NO: 81 - DNA, 59 bp, synthetic construct
  source 1..59: mol_type = other DNA
gactggagttcagacgtgtgctcttccgatctgatcagaatcagtacgcagagagtcgc

SEQ ID NO: 82 - DNA, 59 bp, synthetic construct
  source 1..59: mol_type = other DNA
gactggagttcagacgtgtgctcttccgatcttagcttaatcagtacgcagagagtcgc

SEQ ID NO: 83 - DNA, 59 bp, synthetic construct
  source 1..59: mol_type = other DNA
gactggagttcagacgtgtgctcttccgatctggctagaatcagtacgcagagagtcgc

SEQ ID NO: 84 - DNA, 59 bp, synthetic construct
  source 1..59: mol_type = other DNA
gactggagttcagacgtgtgctcttccgatctcttgtaaatcagtacgcagagagtcgc

SEQ ID NO: 85 - DNA, 63 bp, synthetic construct
  source 1..63: mol_type = other DNA
acactctttccctacacgacgctcttccgatctatcacgagttggtgtcaatacaactcccct

SEQ ID NO: 86 - DNA, 64 bp, synthetic construct
  source 1..64: mol_type = other DNA
acactctttccctacacgacgctcttccgatctcgatgtaagttggtgtcaatacaactcccct

SEQ ID NO: 87 - DNA, 65 bp, synthetic construct
  source 1..65: mol_type = other DNA
acactctttccctacacgacgctcttccgatctttaggctaagttggtgtcaatacaactcccct

SEQ ID NO: 88 - DNA, 66 bp, synthetic construct
  source 1..66: mol_type = other DNA
acactctttccctacacgacgctcttccgatcttgaccagtaagttggtgtcaatacaactcccct

SEQ ID NO: 89 - DNA, 67 bp, synthetic construct
  source 1..67: mol_type = other DNA
acactctttccctacacgacgctcttccgatctacatgtcgtaagttggtgtcaatacaactcccct

SEQ ID NO: 90 - DNA, 68 bp, synthetic construct
  source 1..68: mol_type = other DNA
acactctttccctacacgacgctcttccgatctgccaatacgtaagttggtgtcaatacaactcccct

SEQ ID NO: 91 - DNA, 60 bp, synthetic construct
  source 1..60: mol_type = other DNA
gactggagttcagacgtgtgctcttccgatctcagatccgccagcactttctaaggaatc

SEQ ID NO: 92 - DNA, 60 bp, synthetic construct
  source 1..60: mol_type = other DNA
gactggagttcagacgtgtgctcttccgatctacttgacgccagcactttctaaggaatc

SEQ ID NO: 93 - DNA, 60 bp, synthetic construct
  source 1..60: mol_type = other DNA
gactggagttcagacgtgtgctcttccgatctgatcagcgccagcactttctaaggaatc

SEQ ID NO: 94 - DNA, 60 bp, synthetic construct
  source 1..60: mol_type = other DNA
gactggagttcagacgtgtgctcttccgatcttagcttcgccagcactttctaaggaatc

SEQ ID NO: 95 - DNA, 60 bp, synthetic construct
  source 1..60: mol_type = other DNA
gactggagttcagacgtgtgctcttccgatctggctagcgccagcactttctaaggaatc

SEQ ID NO: 96 - DNA, 60 bp, synthetic construct
  source 1..60: mol_type = other DNA
gactggagttcagacgtgtgctcttccgatctcttgtacgccagcactttctaaggaatc

SEQ ID NO: 97 - DNA, 499 bp, synthetic construct
  source 1..499: mol_type = other DNA
ggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgacgtcctctggctaactagagaacccactgcttactggcttatcgaaatttccctatcagtgatagagattgacatccctatcagtgatagagatactgagcacatcagcaggacgcactgaccagggagacccaagcttgccaccatggtgtacccctacgacgtgcccgactacgccgaattgcctccaaaaaagaagagaaaggtagggatccgaaccatggataagaaatactcaataggcttagatatcggcacaaatagcgtcggatgg

SEQ ID NO: 98 - DNA, 569 bp, synthetic construct
  source 1..569: mol_type = other DNA
ggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgacgtcctctggctaactagagaacccactgcttactggcttatcgaaatttccctatcagtgatagagattgacatccctatcagtgatagagatactgagcacgtgagacccatgccatagcgttgtttagggataacagggtaatactgtccacacaatctgccctggtctccgttttagagctagaaatagcaagttaaaataaggctagtccgttatcaacttgaaaaagtggcaccgagtcggtgcttttttggccggcatggtcccagcctcctcgctggcgccggctgggcaacatgcttcggcatggcgaatgggacgacatcacctcccacaacga

SEQ ID NO: 99 - DNA, 6948 bp, synthetic construct
  source 1..6948: mol_type = other DNA
gacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgacgtcctctggctaactagagaacccactgcttactggcttatcgaaatttccctatcagtgatagagattgacatccctatcagtgatagagatactgagcacatcagcaggacgcactgaccagggagacccaagcttgccaccatggtgtacccctacgacgtgcccgactacgccgaattgcctccaaaaaagaagagaaaggtagggatccgaaccatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 100 - DNA, 4612 bp, synthetic construct
  source 1..4612: mol_type = other DNA
gacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgacgtcctctggctaactagagaacccactgcttactggcttatcgaaatttccctatcagtgatagagattgacatccctatcagtgatagagatactgagcacgtgagacccatgccatagcgttgtttagggataacagggtaatactgtccacacaatctgccctggtctccgttttagagctagaaatagcaagttaaaataaggctagtccgttatcaacttgaaaaagtggcaccgagtcggtgcttttttggccggcatggtcccagcctcctcgctggcgccggctgggcaacatgcttcggcatggcgaatgggacgacatcacctcccacaacgaagactacaccatcgttgaacagtacgaacgtgctgaaggtcgtcactccaccggtgcttaaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcatgatcccctgcgccatcagatccttggcggcaagaaagccatccagtttactttgcagggcttcccaaccttaccagagggcgccccagctggcaattcc

SEQ ID NO: 101 - DNA, 6092 bp, synthetic construct
  source 1..6092: mol_type = other DNA
tcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgattgaaaacgatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttttggggaatataatctagcattacgctagggataacagggtaatatcacgctctagacatacggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatacgtacgatttaaataggcctgactcactatagggagaccggaattccctggcacgacaggtttcccgactggaaagcgggcagtgagcgcaacgcaattaatgtgagttagctcactcattagggaccccgggctttacactttatgcttccggctcgtatgttgtgtggaattgtgagcggataacaatttcacacaggaaacagctatgaccatgattacggattcactggccgtcgttttacaacgtcgtgactggtaaaacccgggcgttacccaacttaatcgccttgcagcacatccccctttcgccagcaggcgtaataaggaaaggatccatgtactatttgaaaaacacaaacttttggatgttcggtttattctttttcttttacttttttatcatgggagcctacttcccgtttttcccgatttggctacatgatatcaaccatatcagcaaaagtgatacgggtattatttttgccgctatttctctgttctcgctattattccaaccgctgtttggtctgctttctgacaaactcggtctacgcaaatacctgctgtggattattaccggcatgttagtgatgtttgcgccgttctttatttttatcttcgggccactgctgcagtacaacattttagtaggatcgattgttggtggtatttatctaggctttagttttaacgccggtgcgccagcagtagaggcatttattgagaaagtcagccggcgcagtaatttcgaatttggtcgcgcgcggatgtttggcagtgttggctgggcgctggttgcctcgattgtcgggatcatgttcaccattaataatcagtttgttttctggctgggctctggcagttgtctcatcctcgccgttttactctttttcgccaaaacggacgcgccctcgagtgccacggttgccaatgcggtaggtgccaaccattcggcatttagccttaagctggcactggaactgttcagacagccaaaactgtggtttttgtcactgtatgttattggcgtttcctccacctacgatgtttttgaccaacagtttgctaatttctttacttcgttctttgctaccggtgaacagggtacccgcgtatttggctacgtaacgacaatgggcgaattacttaacgcctcgattatgttctttgcgccactgatcattaatcgcatcggtgggaagaatgccctgctgctggctggcactattatgtctgtacgtattattggctcatcgttcgccacctcagcgctggaagtggttattctgaaaacgctgcatatgtttgaagtaccgttcctgctggtgggctcctttaaatatattactagtcagtttgaagtgcgtttttcagcgacgatttatctggtcagtttcagcttctttaagcaactggcgatgatttttatgtctgtactggcgggcaatatgtatgaaagcataggtttccaaggcgcttatctggtgctgggtctggtggcgctgggcttcaccttaatttccgtgttcacgcttagcggcccgggcccgctttccctgctgcgtcgtcaggtgaatgaagtcgcttaaaggcc

SEQ ID NO: 102 - DNA, 23 bp, synthetic construct
  source 1..23: mol_type = other DNA
cttgccccacagggcagtaacgg

SEQ ID NO: 103 - DNA, 23 bp, synthetic construct
  source 1..23: mol_type = other DNA
tcagccccacagggcagtaaggg

SEQ ID NO: 104 - DNA, 190 bp, synthetic construct
  source 1..190: mol_type = other DNA
ccactgcttactggcttatcggaagggatcgtcctgaccccgnnnnnnnnnnnnnnnnnnnnnnnccccctccgtggagaaaatttccctatcagtgatagagattgacatccctatcagtgatagagatactgagcacnnnnnnnnnnnnnnnnnnnnnnnnnnnngttttagagctagaaatagcaag

SEQ ID NO: 105 - RNA, 20 nt, synthetic construct
  source 1..20: mol_type = other RNA
gagtccgagcagaagaagaa

SEQ ID NO: 106 - RNA, 32 nt, synthetic construct
  source 1..32: mol_type = other RNA
ttgagtatttcggagtccgagcagaagaagaa

SEQ ID NO: 107 - RNA, 28 nt, synthetic construct
  source 1..28: mol_type = other RNA
cgctctatgagtccgagcagaagaagaa

SEQ ID NO: 108 - RNA, 32 nt, synthetic construct
  source 1..32: mol_type = other RNA
cttgagttgcaagagtccgagcagaagaagaa